(19) United States
(12) Patent Application Publication
(10) Pub. No.: US 2026/0284123 A1
(43) Pub. Date: Sep. 24, 2026

(54) COMPOSITIONS AND METHODS FOR TREATING OR PREVENTING CLOSTRIDIUM DIFFICILE INFECTION, NEUTROPENIA, CARDIOVASCULAR DISEASE, ASTHMA, AND/OR CANCER

(71) Applicant: Vedanta Biosciences, Inc., Cambridge, MA (US)

(72) Inventors: Emily Crossette, Somerville, MA (US); Amanda Prince, Cambridge, MA (US)

(73) Assignee: Vedanta Biosciences, Inc., Cambridge, MA (US)

(21) Appl. No.: 19/471,740

(22) PCT Filed: Apr. 4, 2024
(86) PCT No.: PCT/US2024/023073

§ 371 Date: Oct. 2, 2025

Related U.S. Application Data
(60) Provisional application No. 63/457,055, filed on Apr. 4, 2023.

(51) Int. Cl. A61K 35/742; A61K 38/14; A61P 29/00; A61P 31/04; G01N 33/68

(52) U.S. Cl. CPC A61K 35/742; A61K 38/14; A61P 29/00; A61P 31/04; G01N 33/6893; G01N 2333/4727; G01N 2800/52

(57) ABSTRACT

Provided herein are methods and compositions for treating and/or preventing recurrent Clostridium difficile infection (rCDI), neutropenia, cardiovascular disease (e.g., atherosclerosis and/or stroke), asthma, and/or cancer involving administering to a subject a pharmaceutical composition comprising a purified bacterial mixture.

### RELATED APPLICATION

This application claims the benefit under 35 U.S.C. § 119(e) of U.S. provisional application No. 63/457,055, filed Apr. 4, 2023, the contents of which are incorporated by reference herein in their entirety.

### REFERENCE TO AN ELECTRONIC SEQUENCE LISTING

The contents of the electronic sequence listing (P074570034WO00-SEQ-NTJ.xml; Size: 20,284 bytes; and Date of Creation: Apr. 3, 2024) are herein incorporated by reference in their entirety.

### BACKGROUND

Clostridium difficile infection (CDI) is typically treated with antibiotics, which perturbs the gut microbiome, thereby increasing susceptibility to recurrent CDI (rCDI). For patients who have experienced CDI recurrences, fecal microbiota transplant (FMT) and other donor-derived products have been successful in preventing further recurrences of CDI. The composition and quality attributes of these donor-derived procedures are inherently variable, and they have occasionally resulted in transmission of harmful pathogens from the human donors of the feces (DeFilipp et al., 2019). In addition, the FMT procedure requires extensive and costly donor screening (Craven et al., 2017), and the precise components of the gastrointestinal microbiota that provide resistance against C. difficile remain undetermined (Khanna et al., 2021; McDonald et al., 2018). Neutropenia is a condition characterized by a lower-than-normal abundance of circulating neutrophils. Neutrophils are the most abundant leukocyte (white blood cell) in peripheral circulation, serving as a primary mediator of antibacterial and antiviral immune defense. Subjects with neutropenia are more susceptible to infections and resulting complications, including sepsis and death. Atherosclerosis is a cardiovascular disease in which lesions appear on the artery walls, which may lead to arterial narrowing from plaque buildup. Severe complications of atherosclerosis include coronary artery disease, stroke, peripheral artery disease, and kidney conditions. Stroke is a cardiovascular disease in blockages in blood vessels inhibit blood flow to the brain, or blood vessel rupture leads to blood spilling into the intracranial cavity. Strokes are a significant cause of morbidity and mortality, with complications including hypoxia, deep vein thrombosis, pulmonary embolism, sepsis, and aspiration pneumonia. Asthma is a chronic inflammatory disease of the lungs, characterized by recurring episodes of wheezing, coughing, chest tightness, and shortness of breath. Subjects with asthma are at a higher risk of more severe conditions, including sleep apnea, rhinosinusitis, and gastroesophageal reflux disease (GERD), as well as heart disease and respiratory tract-associated mortality. Cancer is a condition caused by aberrant growth of cells and/or the formation of tumors in a subject, interfering with healthy physiology. Complications of cancer include immune dysregulation, organ failure, and death.

### SUMMARY

Provided herein are compositions and methods for treating and/or preventing conditions including recurrent Clostridium difficile infection (rCDI), neutropenia, cardiovascular disease, asthma, and cancer. It was surprisingly discovered that among subjects administered a combination of 8 bacterial strains, subjects whose fecal calprotectin levels did not increase relative to baseline prior to the administration were less likely to experience C. difficile infection recurrence, while those experiencing recurrence had elevated fecal calprotectin levels following administration of the 8 strains. Maintenance of fecal calprotectin (prevention of substantial increase) thus serves as a clinical outcome and biomarker of effectiveness for preventing C. difficile infection recurrence.

It was also discovered, quite surprisingly, that combinations of 8 bacterial strains, when delivered to the human gastrointestinal tract, modulate the systemic immune response in manners expected to be protective in neutropenia, cardiovascular disease, asthma, and cancer. For example, granulocyte colony stimulating factor (G-CSF), which promotes neutrophil development and mobilization, and consequently mitigates neutrophil deficiency in neutropenia, was maintained in subjects administered such bacterial combinations, whereas subjects administered a placebo control experienced a reduction in G-CSF levels. Additionally, IL-33, which has multiple cardioprotective effects, including reducing atherosclerotic plaque size and promoting tissue repair through M2 macrophage development, was elicited by administration of the bacterial combinations. IL-33 also exhibits additional protective effects in stroke, including limitation of neuroinflammation and preservation of oligodendrocytes. Similarly, fecal calprotectin, a marker of systemic inflammation associated with elevated risk of asthma, was reduced in subjects administered bacterial combinations relative to placebo control. Finally, IL-9, a cytokine with multiple anti-tumor effects, such as induction of innate and adaptive immune responses, as well as induction of tumor cell apoptosis, was induced by administration of such bacterial combinations. Induction of such cytokines, alone or in combination with short-chain fatty acid (SCFA) production, by bacterial combinations as described herein, is expected to provide systemic benefits in addition to local effects such as treating Clostridium difficile (C. difficile) infection or treating and/or preventing C. difficile infection recurrence.

Accordingly, some aspects relate to a method of treating or preventing recurrent Clostridium difficile infection (rCDI) in a subject, the method comprising administering to the subject a composition comprising a purified bacterial mixture, the purified bacterial mixture comprising: (i) a bacterial strain belonging to Clostridium bolteae; (ii) a bacterial strain belonging to Anaerotruncus colihominis; (iii) a bacterial strain belonging to Sellimonas intestinalis; (iv) a bacterial strain belonging to Clostridium symbiosum; (v) a bacterial strain belonging to Blautia producta; (vi) a bacterial strain belonging to Dorea longicatena; (vii) a bacterial strain belonging to Clostridium innocuum; and (viii) a bacterial strain belonging to Flavonifractor plautii, wherein a concentration of fecal calprotectin in a sample of the subject is not substantially increased for at least 1 week after administration of the composition, relative to a baseline concentration of fecal calprotectin prior to administration of the composition. Some aspects relate to a method of treating or preventing recurrent Clostridium difficile infection (rCDI) in a subject, the method comprising administering to the subject a composition comprising a purified bacterial mixture, the purified bacterial mixture comprising: (i) a bacterial strain comprising a 16S rDNA sequence with at least 97% sequence identity to the nucleotide sequence of SEQ ID NO: 1; (ii) a bacterial strain comprising a 16S rDNA sequence with at least 97% sequence identity to the nucleotide sequence of SEQ ID NO: 2; (iii) a bacterial strain comprising a 16S rDNA sequence with at least 97% sequence identity to the nucleotide sequence of SEQ ID NO: 3; (iv) a bacterial strain comprising a 16S rDNA sequence with at least 97% sequence identity to the nucleotide sequence of SEQ ID NO: 4; (v) a bacterial strain comprising a 16S rDNA sequence with at least 97% sequence identity to the nucleotide sequence of SEQ ID NO: 5; (vi) a bacterial strain comprising a 16S rDNA sequence with at least 97% sequence identity to the nucleotide sequence of SEQ ID NO: 6; (vii) a bacterial strain comprising a 16S rDNA sequence with at least 97% sequence identity to the nucleotide sequence of SEQ ID NO: 7; and (viii) a bacterial strain comprising a 16S rDNA sequence with at least 97% sequence identity to the nucleotide sequence of SEQ ID NO: 8, wherein a concentration of fecal calprotectin in a sample of the subject is not substantially increased for at least 1 week after administration of the composition, relative to a baseline concentration of fecal calprotectin prior to administration of the composition.

In some embodiments, the concentration of fecal calprotectin in the sample of the subject does not exceed 140%, 130%, 120%, or 110% of the baseline concentration of fecal calprotectin in the subject. In some embodiments, the concentration of fecal calprotectin is not substantially increased over a period of at least 2, 3, 4, 5, 6, 7, or 8 weeks following administration of the composition. In some embodiments, the baseline concentration of fecal calprotectin is a fecal calprotectin concentration in a baseline sample of the subject obtained 48 hours or fewer prior to administration of the composition. In some embodiments, the method further comprises measuring fecal calprotectin in one or more samples of the subject after administration of the composition. In some embodiments, the method further comprises identifying the subject as having an elevated concentration of fecal calprotectin, if the level of fecal calprotectin is increased relative to the baseline concentration, and administering one or more additional doses of the composition to the subject. In some embodiments, the elevated concentration of fecal calprotectin is at least 150%, at least 175%, at least 200%, at least 250%, or at least 300% relative to the baseline concentration of fecal calprotectin in the subject.

Some aspects relate to a method of treating and/or preventing a condition in a subject, the method comprising administering to the subject a composition comprising a purified bacterial mixture, the purified bacterial mixture comprising: (i) a bacterial strain belonging to Clostridium bolteae; (ii) a bacterial strain belonging to Anaerotruncus colihominis; (iii) a bacterial strain belonging to Sellimonas intestinalis; (iv) a bacterial strain belonging to Clostridium symbiosum; (v) a bacterial strain belonging to Blautia producta; (vi) a bacterial strain belonging to Dorea longicatena; (vii) a bacterial strain belonging to Clostridium innocuum; and (viii) a bacterial strain belonging to Flavonifractor plautii, wherein the condition is selected from the group consisting of neutropenia, cardiovascular disease, asthma, and cancer. Some aspects relate to a method of treating and/or preventing a condition in a subject, the method comprising administering to the subject a composition comprising a purified bacterial mixture, the purified bacterial mixture comprising: (i) a bacterial strain comprising a 16S rDNA sequence with at least 97% sequence identity to the nucleotide sequence of SEQ ID NO: 1; (ii) a bacterial strain comprising a 16S rDNA sequence with at least 97% sequence identity to the nucleotide sequence of SEQ ID NO: 2; (iii) a bacterial strain comprising a 16S rDNA sequence with at least 97% sequence identity to the nucleotide sequence of SEQ ID NO: 3; (iv) a bacterial strain comprising a 16S rDNA sequence with at least 97% sequence identity to the nucleotide sequence of SEQ ID NO: 4; (v) a bacterial strain comprising a 16S rDNA sequence with at least 97% sequence identity to the nucleotide sequence of SEQ ID NO: 5; (vi) a bacterial strain comprising a 16S rDNA sequence with at least 97% sequence identity to the nucleotide sequence of SEQ ID NO: 6; (vii) a bacterial strain comprising a 16S rDNA sequence with at least 97% sequence identity to the nucleotide sequence of SEQ ID NO: 7; and (viii) a bacterial strain comprising a 16S rDNA sequence with at least 97% sequence identity to the nucleotide sequence of SEQ ID NO: 8, wherein the condition is selected from the group consisting of neutropenia, cardiovascular disease, asthma, and cancer.

In some embodiments, the subject has neutropenia. In some embodiments, the neutropenia results from cancer treatment. In some embodiments, the subject has a cardiovascular disease. In some embodiments, the subject has atherosclerosis. In some embodiments, the subject has had a stroke. In some embodiments, the subject has asthma. In some embodiments, the subject has cancer. In some embodiments, the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

In some embodiments, the composition induces neutrophil differentiation in the subject. In some embodiments, the composition increases circulating neutrophil abundance in the subject. In some embodiments, the composition inhibits infiltration of neutrophils into the intestinal lumen of the subject. In some embodiments, the composition induces proliferation and/or accumulation of regulatory T cells (Tregs) in the subject. In some embodiments, the Tregs are ST2+ Tregs. In some embodiments, the composition promotes tissue repair in the subject. In some embodiments, the composition induces production of a protein selected from the group consisting of calprotectin, IL-5, IL-9, IL-10, IL-12(p70), IL20, IL-23, IL-28A, IL-33, thymic stromal lymphopoietin (TSLP), thrombopoietin (TPO), macrophage inflammatory protein-1 alpha (MIP-1α), monocyte chemotactic protein 3 (MCP3), leukemia inhibitory factor (LIF), interferon-gamma (IFN-γ), fractalkine, and eotaxin-3, in the subject. In some embodiments, the composition induces production of calprotectin in the subject.

In some embodiments, the purified bacterial mixture consists of: (i) a bacterial strain comprising a 16S rDNA sequence with at least 99% sequence identity to the nucleotide sequence of SEQ ID NO: 1; (ii) a bacterial strain comprising a 16S rDNA sequence with at least 99% sequence identity to the nucleotide sequence of SEQ ID NO: 2; (iii) a bacterial strain comprising a 16S rDNA sequence with at least 99% sequence identity to the nucleotide sequence of SEQ ID NO: 3; (iv) a bacterial strain comprising a 16S rDNA sequence with at least 99% sequence identity to the nucleotide sequence of SEQ ID NO: 4; (v) a bacterial strain comprising a 16S rDNA sequence with at least 99% sequence identity to the nucleotide sequence of SEQ ID NO: 5; (vi) a bacterial strain comprising a 16S rDNA sequence with at least 99% sequence identity to the nucleotide sequence of SEQ ID NO: 6; (vii) a bacterial strain comprising a 16S rDNA sequence with at least 99% sequence identity to the nucleotide sequence of SEQ ID NO: 7; and (viii) a bacterial strain comprising a 16S rDNA sequence with at least 99% sequence identity to the nucleotide sequence of SEQ ID NO: 8.

In some embodiments, the bacterial strains are lyophilized or spray-dried. In some embodiments, one or more of the bacterial strains are in spore form. In some embodiments, the composition is a pharmaceutical composition comprising the purified bacterial mixture and a pharmaceutically acceptable excipient. In some embodiments, the pharmaceutical composition is administered orally. In some embodiments, the pharmaceutical composition is administered rectally. In some embodiments, the pharmaceutical composition is formulated for delivery to the intestine. In some embodiments, the pharmaceutical composition is formulated for delivery to the colon. In some embodiments, the pharmaceutical composition comprises one or more enteric polymers.

In some embodiments, the pharmaceutical composition is administered to the subject as multiple doses over multiple days. In some embodiments, the subject is administered about 1.6×10⁹ colony-forming units (CFUs) of the bacterial strains per day. In some embodiments, the subject is administered a total of about 2.2×10¹⁰ colony-forming units (CFUs) of the bacterial strains. In some embodiments, the subject is administered about 8.0×10⁹ colony-forming units (CFUs) of the bacterial strains per day. In some embodiments, the subject is administered about 1.1×10¹¹ colony-forming units (CFUs) of the bacterial strains.

In some embodiments, the method comprises administering an antibiotic to the subject prior to administration of the pharmaceutical composition. In some embodiments, the antibiotic is vancomycin. In some embodiments, the subject is a human.

These and other aspects of the disclosure, as well as various embodiments thereof, will become more apparent in reference to the drawings and detailed description of the disclosure.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF THE DRAWINGS

FIG. 1 shows the recruitment, dosing, monitoring, and endpoints of a double-blind placebo-controlled Phase 2 clinical trial in which a combination of 8 live bacterial strains (VE303) was administered to subjects at risk of recurrent C. difficile infection (rCDI).

FIGS. 2A-2E show fecal calprotectin levels over time in subjects administered a combination of 8 live bacterial strains or placebo control. FIG. 2A shows data from subjects administered a placebo control. FIG. 2B shows data from subjects administered VE303 at a dose of 2.2×10¹⁰ CFU (VE303 low-dose). FIG. 2C shows data from subjects administered VE303 at a dose of 1.1×10¹¹ CFU (VE303 high-dose). Thin lines show individual time courses, while thick lines show average calprotectin levels among subjects with on-study CDI recurrence, or among subjects without recurrence. FIG. 2D shows log 2 fold change from day 1 to 14, stratified by treatment group. FIG. 2E shows correlations between fecal calprotectin and serum levels for each of the indicated cytokines. Top row (left to right): eotaxin-3, G-CSF, GROa (CXCL1), IL-1RA, and IL-4. Bottom row (left to right): IL-5, IL-6, IL-8, MIG/CXCL9, and TNF-α. Results shown for linear mixed effects model (log₁₀(Cytokine) ~log₁₀(fecal calprotectin)+(1|Subject)), including data from all timepoints where both cytokine concentrations and fecal calprotectin were measured in a subject.

FIGS. 3A-3H show serum cytokine levels in subjects administered a combination of 8 live bacterial strains or placebo control. FIG. 3A shows prevalence of the indicated cytokines in subjects administered the VE303 high dose (1.1×10¹¹ CFU), VE303 low dose (2.2×10¹⁰ CFU), or a placebo control, from all timepoints. FIG. 3B shows log 2 fold change (L2FC) of each of the indicated cytokines over time (results from linear mixed effects showing significant (p<0.1) alterations over time). FIGS. 3C and 3D shows log₁₀ cytokine concentration for each of the indicated cytokines over time following administration of the VE303 high dose (1.1×10¹¹ CFU), VE303 low dose (2.2×10¹⁰ CFU), or a placebo control. FIG. 3E shows L2FC of IL-5 over time. FIG. 3F shows L2FC of eotaxin-3 over time. FIG. 3G shows L2FC of G-CSF over time. FIG. 3H shows L2FC of IL-17F over time.

### DETAILED DESCRIPTION

Provided herein are compositions and methods for treating and/or preventing conditions including recurrent Clostridium difficile infection (rCDI), neutropenia, cardiovascular disease (e.g., atherosclerosis and/or stroke), asthma, and cancer. It was surprisingly discovered that maintenance of fecal calprotectin levels was indicative of nonrecurrence of C. difficile infection, following administration of a combination of 8 bacterial strains, at both low and high doses of the combination. This finding suggests that monitoring of fecal calprotectin levels is useful, for instance, as a measure of clinical efficacy of the combination of bacterial strains. For example, fecal calprotectin levels may be monitored following administration of a composition, with lack of a substantial increase in fecal calprotectin indicating the subject is at reduced risk of CDI recurrence, while an increase in fecal calprotectin levels (relative to a baseline level) indicates that the subject is at elevated risk of recurrence and/or experiencing more severe symptoms. Such detection of elevated fecal calprotectin levels allows for improved evaluation of clinical outcomes and earlier intervention, such as by administering additional doses of the combination of bacterial strains and/or antibiotics to prevent or mitigate C. difficile recurrence, where increased risk of recurrence is identified.

It was also discovered, quite surprisingly, that a combination of 8 bacterial strains induced cytokines involved in granulocyte production, tissue repair, reduction of inflammation, and/or anti-tumor immunity. Such cytokine induction provides additional systemic benefits, including modulation of the immune response and tissue repair in manners that alleviate or prevent neutropenia (e.g., by promoting neutrophil generation through induction of granulocyte colony stimulating factor (G-CSF)), cardiovascular disease (e.g., by reducing inflammation and promoting tissue repair through induction of IL-33), asthma (e.g., by reducing inflammation), and/or cancer (e.g., by promoting anti-tumor immune responses through induction of IL-9).

Neutropenia is a condition characterized by a reduction in the number of circulating neutrophils, which mediate protection from bacterial and viral infections. Accordingly, a subject having neutropenia may be more susceptible to bacterial and/or viral infection or experience more severe infection. Some aspects of the methods described herein are based, at least in part, on the recognition that bacterial combinations elicit cytokines that may promote hematopoiesis and/or reduce the risk of developing neutropenia. For example, levels of circulating G-CSF, which stimulates granulocyte (neutrophil, basophil, eosinophil) production and is commonly used for treating neutropenia, were maintained in human subjects administered a combination of 8 live bacterial strains, whereas G-CSF levels were substantially reduced in subjects administered a placebo control. Without wishing to be bound by any particular theory, it is believed that the compositions and methods described herein are useful for treating and/or preventing neutropenia by inducing G-CSF, which enhances proliferation of granulocyte lineages from myeloblast to myelocyte, promotes differentiation of neutrophils, and accelerates their maturation, allowing rapid and continuous elevation of neutrophils in blood. See, e.g., Theyab et al., Hematology. 2021. 26(1):628-636.

Additional embodiments of the methods described herein are based, at least in part, on the recognition that the composition of the gut microbiome affects the hematopoiesis, which consequently may increase or decrease the risk of developing neutropenia. For example, neutropenia often occurs following depletion of bacteria from the gut by antibiotic therapy. See, e.g., Yan et al., Blood. 2018. 132(6):559-564. Neutropenia is also common in cancers including leukemia and preceded by altered cross-talk between the gut microbiome and the host immune system. See, e.g., Rashidi et al., Blood Adv. 5(20):3937-3950. Similarly, neutropenia often occurs after chemotherapy for cancer treatment.

Atherosclerosis is characterized by accumulation of plaque on arterial walls. Strokes are charactered by blockages of blood vessels which limit the flow of blood to the brain (ischemic) or ruptured blood vessels leading to the flow of blood into the intracranial cavity. Some aspects of the methods described herein are based, at least in part, on the recognition that bacterial combinations elicit cytokines that alleviate cardiovascular disease, such as atherosclerosis and stroke. For example, cytokines IL-5, IL-9, and IL-33, which are associated with tissue repair, were elevated in subjects administered a combination of 8 live bacterial strains, whereas levels of these cytokines were substantially reduced in subjects administered a placebo control. Without wishing to be bound by any particular theory, it is believed that compositions and methods described herein are useful for treating and/or preventing cardiovascular diseases such as atherosclerosis and stroke, by inducing cytokines including IL-33, which exerts protective effects through multiple mechanisms.

The IL-33 signaling pathway, or IL-33/ST2 axis, in regulatory T cells (Treg) enhances transforming growth factor (TGF)-β1-mediated differentiation of Treg cells and provides a signal for Treg cells in inflamed tissues. In particular, IL-33 binds to the heterodimer of ST2 and IL-1 receptor accessory protein (IL-1RAP), which recruits intracellular MyD88. MyD88 recruitment consequently promotes FoxP3 and GATA3 expression, while also promoting Treg function and proliferation by enhancing TGF-β1-mediated differentiation though a p38-dependent mechanism. See, e.g., Griesenauer et al., Front Immunol. 2017. 24(8):475. MyD88 recruitment also recruits IL-1R-associated kinases (IRAK1, IRAK2, IRAK3, IRAK4) and TRAF6, leading to activation of either the NF-1B or AP-1 (via MAPK) pathways. See, e.g., Chang et al., Transl Perioper & Pain Med, 2016. 1(2):24-36. Stimulation of IL-33 promotes development of a tissue repair phenotype in Tregs, which mediate tolerance to inflammation, negatively regulate potentially damaging inflammatory cells (e.g., neutrophils and macrophages), and produce growth factors and mediators including amphiregulin (AREG), keratinocyte growth factor (KGF), and/or TGF-β. With respect to cardiovascular function, downstream of effects of IL-33 signaling include reduced cell death of cardiomyocytes, expansion of M2 macrophages, a reparative macrophage subset, and induction of IL-5 production, which promotes generation of cardioprotective antioxidized low-density lipoprotein (ox-LDL) antibodies. See, e.g., Chen et al., Cell Physiol Biochem. 2018. 49(1):349-358; Miller et al., J Exp Med. 2008. 205:339-346. IL-33 signaling in neuronal macrophages (microglia) induces a similar protective response, including phagocytosis of extracellular matrix (regulating synapse remodeling), clearance of A3 (mitigating neuroinflammation), and preserving oligodendrocytes and oligodendrocyte precursor cells, thereby mitigating the effects of ischemic injury in stroke. Xie et al., Stroke. 2021. 52(6):2150-2161.

Additional embodiments of the methods described herein are based, at least in part, on the recognition that the composition of the gut microbiome affects the development of cardiovascular disease. For example, reduction in butyrate-producing bacteria decreases mucosal barrier integrity, leading to increased infiltration of pro-inflammatory lipopolysaccharide (LPS) into circulation, a risk factor for cardiovascular disease. Additionally, dietary supplementation with butyrate markedly reduces cardiovascular disease. See, e.g., Shen et al., Front Cardiovasc Med. 2021. 8:668532.

Asthma is a condition characterized by airway inflammation and obstruction. Some aspects of the methods described herein are based, at least in part, on the recognition that bacterial combinations elicit cytokines that alleviate or prevent asthma symptoms, and/or decrease the risk of developing asthma. For example, fecal calprotectin is a biomarker of intestinal inflammation that is associated with risk of asthma. See, e.g., Orivuori et al., Clin Exp Allergy. 2015. 45(5):928-939. In subjects administered a combination of 8 live bacterial strains, inflammation was substantially reduced, as evidenced by lower fecal calprotectin levels, compared to subjects administered a placebo control. Without wishing to be bound by any particular theory, it is believed that compositions and methods described herein are useful for treating and/or preventing asthma by reducing systemic inflammation (e.g., by inducing regulatory T cell proliferation and secretion of anti-inflammatory IL-10), as evidenced by reduced fecal calprotectin compared to subjects administered a placebo control.

Additional embodiments of the methods described herein are based, at least in part, on the recognition that the composition of the gut microbiome affects the physiology of the lungs, which consequently may increase or decrease the risk of developing or worsening of asthma. For example, depletion of endogenous microbiota by vancomycin treatment decreases the abundance of short-chain fatty acid (SCFA)-producing bacteria and circulating SCFA levels, but supplementation of SCFAs reduces asthma susceptibility in mouse models. Such SCFAs inhibit T cells and dendritic cells associated with Th2 responses, suggesting a role for bacterial SCFA production in mitigating asthma. See, e.g., Cait et al., Mucosal Immunol. 2018. 1193:785-795. More generally, gut-lung communication is thought to occur through activation or inhibition of immune cells by gut microbes or their secreted metabolites, as well as through secretion of metabolites by microbes that then enter peripheral circulation. See, e.g., Cobos-Uribe and Rebuli, Curr Allergy Asthma Rep. 2023. 23(2):67-76.

Cancer is characterized by aberrant cellular proliferation, which may lead to formation of tumors in a subject. Such uncontrolled proliferation, including tumor formation, interferes with normal physiology. Some aspects of the methods described herein are based, at least in part, on the recognition that bacterial combinations elicit cytokines with anti-tumor benefits. For example, IL-9, which mediates multiple aspects of anti-tumor immunity, was observed to be elevated in subjects administered a combination of 8 live bacterial strains, whereas IL-9 levels were substantially reduced in subjects administered a placebo control. Without wishing to be bound by any particular theory, it is believed that compositions and methods described herein are useful for treating and/or preventing cancer by inducing IL-9, which exerts anti-tumor activity through multiple mechanisms, including inducing innate immune responses, inducing adaptive immune responses, and promoting apoptosis of tumor cells. See, e.g., Zheng and Lu, Hum Vaccin Immunother. 2020. 16(10):2333-2340.

Some Clostridia strains, such as those of the compositions and methods provided herein, effectively metabolize primary bile acids to secondary bile acids and also produce substantial amounts of SCFAs. The metabolic activities of these and other bacterial strains are described, for instance, in PCT Publication No. WO 2020/037271, which is incorporated herein by reference in its entirety. See, e.g., Example 5 of PCT Publication No. WO 2020/037271, demonstrating the ability of Clostridium bolteae, Anaerotruncus colihominis, Sellimonas intestinalis, Clostridium symbiosum, Blautia producta, Dorea longicatena, Clostridium innocuum, and Flavonifractor plautii to increase levels of secondary bile acids in a subject when administered after antibiotic treatment, which reduces or clears other gut-resident microflora (e.g., FIGS. 32-33). See, also, Example 6 of PCT Publication No. WO 2020/037271, demonstrating the ability of the same bacterial strains to produce SCFAs such as acetate, propionate, and butyrate, when administered to subjects under similar conditions (e.g., FIGS. 37-38).

The disclosed compositions and methods are not limited in its application to the details of construction and the arrangement of components set forth in the following description or illustrated in the drawings. The disclosed compositions and methods are capable of other embodiments and of being practiced or of being carried out in various ways.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Some aspects of the disclosure relate to methods for treating or preventing recurrent C. difficile infection (rCDI) in a subject, the method comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating or preventing recurrent C. difficile infection (rCDI) in a subject, the method comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

In some embodiments of methods for treating or preventing rCDI in a subject, the concentration of fecal calprotectin in a sample of the subject is not substantially increased for at least 1 week after administration of the bacterial strains, relative to a baseline concentration of fecal calprotectin prior to administration of the bacterial strains.

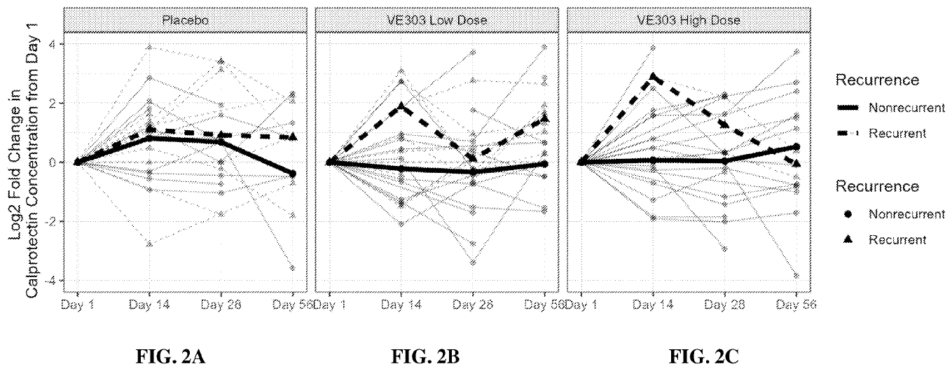

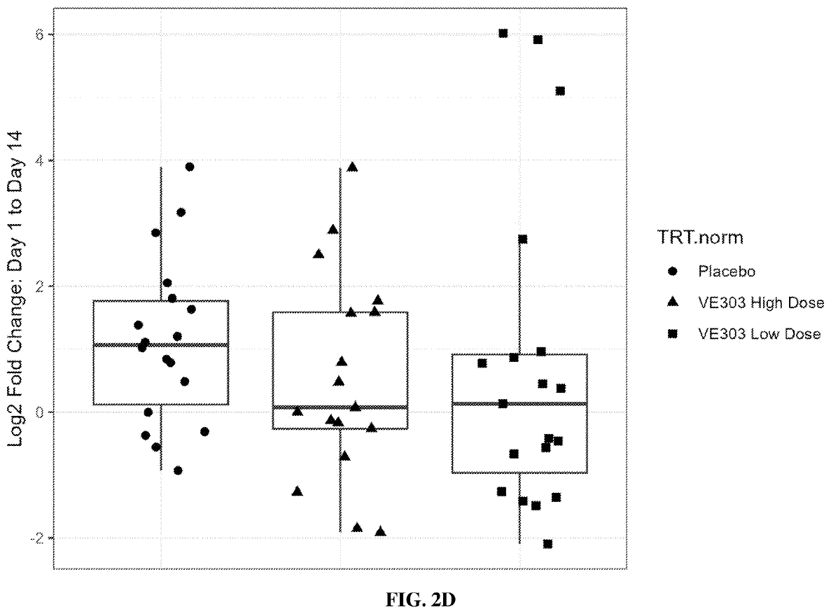

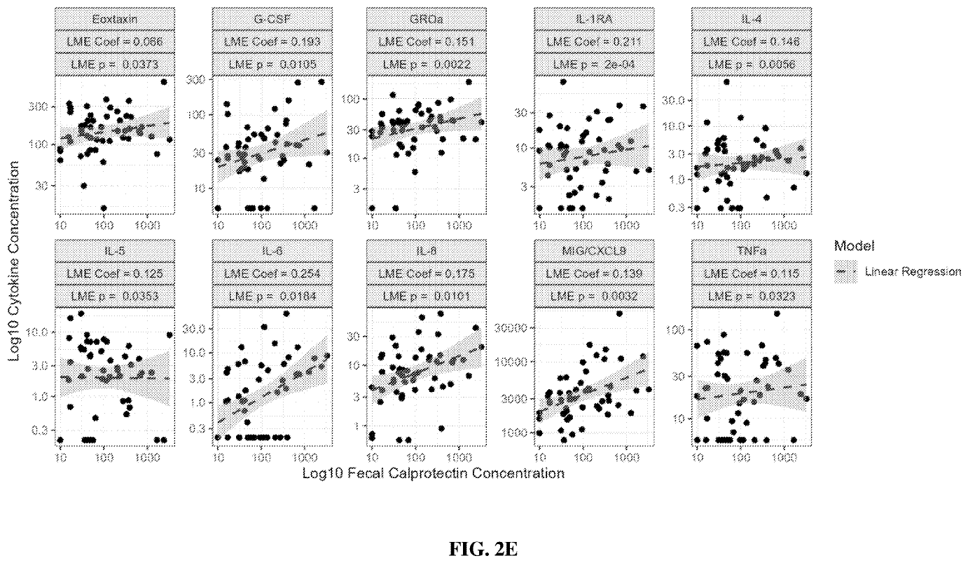

Calprotectin is the major component of neutrophil cytosol, and its presence in fecal samples indicates the presence of neutrophils in the intestinal lumen, and by extension intestinal inflammation. Fecal calprotectin levels were reduced in subjects administered an 8-strain bacterial mixture, relative to subjects who received a placebo (FIG. 2D), with maintenance of fecal calprotectin at levels similar to baseline being associated with non-recurrence of C. difficile infection (FIGS. 2B, 2C). Without wishing to be bound by a particular theory, it is expected that maintenance of fecal calprotectin levels is indicative of reduced risk of C. difficile infection recurrence, whereas an increase in fecal calprotectin relative to baseline indicates an elevated risk of recurrence. Administering an 8-strain bacterial mixture to a subject such that fecal calprotectin levels are not substantially increased therefore allows for a reduction in the likelihood of C. difficile infection recurrence.

After administration of a composition, fecal calprotectin levels are said to be “not substantially increased” relative to baseline, where a concentration of calprotectin detected in a fecal sample from the subject after the administration is 140% or less compared to the baseline concentration of calprotectin in a fecal sample from the subject prior to administration. In some embodiments, the fecal calprotectin concentration in a sample from the subject does not exceed 140%, 135%, 130%, 125%, 120%, 115%, 110%, or 105% relative to the subject's baseline fecal calprotectin concentration. In some embodiments, the fecal calprotectin concentration in a sample from the subject is within 60% to 140%, 70% to 130%, 80% to 120%, or 90% to 110% relative to the subject's baseline fecal calprotectin concentration over a period of time. In some embodiments, the fecal calprotectin concentration in a sample from the subject is within 70% to 140%, 70% to 130%, 70% to 120%, or 70% to 110% relative to the subject's baseline fecal calprotectin concentration over a period of time. In some embodiments, the fecal calprotectin concentration in a sample from the subject is not substantially increased over a period of at least 1, 2, 3, 4, 5, 6, 7, 8, 9, 10, 11, 12, 13, 14, 15, 16, 17, 18, 19, or 20 weeks following administration of the composition. In some embodiments, the baseline concentration of fecal calprotectin is defined as the concentration measured in a fecal sample obtained from the subject within 48 hours or fewer, 36 hours or fewer, 24 hours or fewer, 12 hours or fewer, 8 hours or fewer, 6 hours or fewer, or 4 hours or fewer prior to administration. Where a subject is administered bacterial strains over multiple days, the timing of fecal sample collection prior to administration and/or the duration over which fecal calprotectin levels are maintained or not substantially increased is calculated starting from administration of the first dose of bacterial strains.

In some embodiments, the method further comprises measuring the calprotectin concentration(s) in one or more fecal samples obtained from the subject after administration of the composition. In some embodiments, fecal samples are obtained from the subject at regular intervals for at least 1, 2, 3, 4, 5, 6, 7, 8, 9, 10, 11, 12, 13, 14, 15, 16, 17, 18, 19, or 20 weeks, and assayed to determine calprotectin concentrations. In some embodiments, fecal samples are collected weekly and assayed to determine calprotectin concentrations.

In some embodiments, the method further comprises identifying the subject as being at elevated risk for recurrence of C. difficile infection and administering a therapy to the subject. In some embodiments, the method further comprises identifying the subject as having an elevated fecal calprotectin concentration in a sample from the subject relative to the baseline fecal calprotectin concentration and administering a therapy to the subject. In some embodiments, the elevated fecal calprotectin concentration in the sample is at least 150%, at least 160%, at least 170%, at least 175%, at least 180%, at least 190%, at least 200%, at least 225%, at least 250%, at least 275%, at least 300%, at least 350%, at least 400%, at least 500%, at least 600%, at least 700%, or at least 800% of the baseline fecal calprotectin concentration. In some embodiments, the elevated fecal calprotectin concentration is 200% to 800%, 200% to 700%, 200% to 600%, 200% to 500%, 200% to 400%, or 200% to 300% of the baseline fecal calprotectin concentration. In some embodiments, the elevated fecal calprotectin concentration is 200% to 300%, 300% to 400%, 400% to 500%, 500% to 600%, 600% to 700%, or 700% to 800%.

In some embodiments, the therapy is one or more additional doses of the composition comprising bacterial strains. In some embodiments, the therapy is an antibiotic. In some embodiments, the therapy comprises one or more doses of an antibiotic, followed by one or more additional doses of the composition comprising bacterial strains. In some embodiments, the antibiotic is selected from vancomycin, kanamycin, gentamicin, colistin, metronidazole, clindamycin, fidaxomicin, penicillin, streptomycin, and cefoperazone. In some embodiments, the antibiotic is vancomycin.

Some aspects of the disclosure relate to methods for treating and/or preventing neutropenia in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing neutropenia in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Some aspects of the disclosure relate to methods for treating and/or preventing cardiovascular disease in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing cardiovascular disease in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Some aspects of the disclosure relate to methods for treating and/or preventing atherosclerosis in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing atherosclerosis in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Some aspects of the disclosure relate to methods for treating and/or preventing stroke in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing stroke in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Some aspects of the disclosure relate to methods for treating and/or preventing asthma in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing asthma in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Some aspects of the disclosure relate to methods for treating and/or preventing cancer in a subject comprising administering to the subject a composition comprising a purified bacterial mixture comprising one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. Some aspects of the disclosure relate to methods for treating and/or preventing cancer in a subject comprising administering to the subject a composition comprising a purified bacterial mixture comprising one or more bacterial strains comprising 16S rDNA sequences having at least 97% sequence identity with the nucleic acid sequences selected from the group consisting of SEQ ID NOs: 1-8.

Aspects of the present disclosure provide compositions comprising mixtures of bacterial strains. Some embodiments of the bacterial strains are described for instance in PCT Publication No. WO 2017/218680, which is incorporated herein by reference in its entirety.

In some embodiments of the compositions provided herein, the composition includes one or more of the bacterial strains provided in Table 1. In some embodiments of the compositions provided herein, the composition includes one or more of the following bacterial strains: Clostridium bolteae (Lachnoclostridium bolteae, Enterocloster bolteae), Anaerotruncus colihominis, Sellimonas intestinalis (Drancourtella massiliensis, Ruminococcus torques, Eubacterium fissicatena), Clostridium symbiosum (Lachnoclostridium symbiosum), Blautia producta (Blautia sp001304935), Dorea longicatena, Clostridium innocuum (Erysipelotrichaceae innocuum, Eubacterium innocuum, Absiella innocuum, Longicatena innocuum, Erysipelotrichaceae bacterium), and Flavonifractor plautii (Clostridium orbiscindens, Subdolinogranulum spp). In some embodiments, the compositions described herein comprise at least 2, at least 3, at least 4, at least 5, at least 6, at least 7, or at least 8 bacterial strains (e.g., purified bacterial strains) listed in Table 1.

As will be appreciated by one of ordinary skill in the art, a bacterial strain may be closely related to one or more bacterial species. Alternatively or in addition, a bacterial strain may be referred to by one or more bacterial species names, based on changing nomenclature and phylogenetic classification. In some embodiments, the composition includes Clostridium bolteae. In some embodiments, the bacterial strain referred to as Clostridium bolteae and having a 16S rDNA sequence comprising the nucleic acid sequence of SEQ ID NO: 1 may also be referred to, for example, as Lachnoclostridium bolteae or Enterocloster bolteae. In some embodiments, the composition includes Anaerotruncus colihominis. In some embodiments, the bacterial strain referred to as Anaerotruncus colihominis has a 16S rDNA sequence comprising the nucleic acid sequence of SEQ ID NO: 2. In some embodiments, the composition includes Sellimonas intestinalis. In some embodiments, the bacterial strain referred to as Sellimonas intestinalis and having a 16S rDNA sequence comprising the nucleic acid sequence of SEQ ID NO: 3 may also be referred to, for example, as Drancourtella massiliensis, Eubacterium fissicatena, or Ruminococcus torques. In some embodiments, the composition includes Clostridium symbiosum. In some embodiments, the bacterial strain referred to as Clostridium symbiosum and having a 16S rDNA sequence comprising the nucleic acid sequence of SEQ ID NO: 4 may also be referred to, for example, as Lachnoclostridium symbiosum. In some embodiments, the composition includes Blautia producta. In some embodiments, the bacterial strain referred to as Blautia producta and having a 16S rDNA sequence comprising the nucleic acid sequence of SEQ ID NO: 5 may also be referred to, for example, as Blautia sp001304935. In some embodiments, the composition includes Dorea longicatena. In some embodiments, the bacterial strain referred to as Dorea longicatena has a 16S rDNA sequence comprising the nucleic acid sequence of SEQ ID NO: 6. In some embodiments, the composition includes Clostridium innocuum. In some embodiments, the bacterial strain referred to as Clostridium innocuum and having a 16S rDNA sequence comprising the nucleic acid sequence of SEQ ID NO: 7 may also be referred to, for example, as Erysipelotrichaceae bacterium, Erysipelotrichaceae innocuum, Eubacterium innocuum, Absiella innocuum, and Longicatena innocuum. In some embodiments, the composition includes Flavonifractor plautii. In some embodiments, the bacterial strain referred to as Flavonifractor plautii and having a 16S rDNA sequence comprising the nucleic acid sequence of SEQ ID NO: 8 may also be referred to, for example, as Subdolinogranulum spp. or Clostridium orbiscindens.

In some aspects, the composition comprises a purified bacterial mixture comprising one or more (e.g., 1, 2, 3, 4, 5, 6, 7, or 8) bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. In some aspects, the composition comprises a purified bacterial mixture consisting of one or more (e.g., 1, 2, 3, 4, 5, 6, 7, or 8) bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii.

In some aspects, the composition comprises a purified bacterial mixture comprising Clostridium bolteae, Anaerotruncus colihominis, Sellimonas intestinalis, Clostridium symbiosum, Blautia producta, Dorea longicatena, Clostridium innocuum, and Flavonifractor plautii. In some aspects, the composition comprises a purified bacterial mixture consisting of Clostridium bolteae, Anaerotruncus colihominis, Sellimonas intestinalis, Clostridium symbiosum, Blautia producta, Dorea longicatena, Clostridium innocuum, and Flavonifractor plautii.

In some aspects, the compositions and methods provided herein alleviate and/or reduce the likelihood of recurrent C. difficile infection in a subject. In some aspects, the compositions and methods provided herein alleviate and/or reduce the likelihood of neutropenia in a subject. In some aspects, the compositions and methods provided herein alleviate and/or reduce the likelihood of cardiovascular disease in a subject. In some aspects, the compositions and methods provided herein alleviate and/or reduce the likelihood of atherosclerosis in a subject. In some aspects, the compositions and methods provided herein alleviate and/or reduce the likelihood of stroke in a subject. In some aspects, the compositions and methods provided herein alleviate and/or reduce the likelihood of asthma in a subject. In some aspects, the compositions and methods provided alleviate and/or reduce the likelihood of cancer in a subject. In some embodiments, likelihood is assessed over a specific period of time. In some embodiments, likelihood is assessed over 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, likelihood is assessed over 1, 2, 3, 4, 5, 6, 7, 8, 9, 10, 11, or 12 months. In some embodiments, likelihood is assessed over 1, 2, 3, 4, 5, 6, 7, 8, 9, 10, 11, 12, 13, 14, 15, 16, 17, 18, 19, or 20 years.

In some aspects, the compositions and methods provided herein alleviate and/or reduce the incidence of recurrent C. difficile infection in a subject. In some aspects, the compositions and methods provided herein alleviate and/or reduce the incidence of neutropenia in a subject. In some aspects, the compositions and methods provided herein alleviate and/or reduce the incidence of one or more symptoms of cardiovascular disease in a subject. In some aspects, the compositions and methods provided herein alleviate and/or reduce the incidence of one or more symptoms of atherosclerosis in a subject. In some aspects, the compositions and methods provided herein alleviate and/or reduce the incidence of stroke in a subject. In some aspects, the compositions and methods provided herein alleviate and/or reduce the incidence of one or more symptoms of asthma in a subject. In some aspects, the compositions and methods provided herein alleviate and/or reduce the likelihood of asthma in a subject. In some aspects, the compositions and methods provided alleviate and/or reduce the incidence of cancer in a subject. In some embodiments, incidence is assessed over a specific period of time. In some embodiments, incidence is assessed over 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, incidence is assessed over 1, 2, 3, 4, 5, 6, 7, 8, 9, 10, 11, or 12 months. In some embodiments, incidence is assessed over 1, 2, 3, 4, 5, 6, 7, 8, 9, 10, 11, 12, 13, 14, 15, 16, 17, 18, 19, or 20 years.

The exemplary bacterial strains of compositions disclosed herein can also be identified by their 16S rRNA sequences or 16S rRNA coding sequences (16S rDNA sequences of SEQ ID NOs: 1-8).

Identifying bacteria by their sequences furthermore allows for the identification of additional bacterial strains that are identical or highly similar to the exemplified bacteria. For instance, the 16S rRNA coding sequences (16S rDNA sequences) of bacterial strains were used to identify the closest relative (based on percent identity) through whole genome sequencing and by comparing these sequences with 16S databases (Table 1). In addition, based on whole genome sequencing (WGS) and comparing of the whole genome to whole genome (WG) databases, the bacterial strains having 16S rRNA coding (16S rDNA) sequences provided by SEQ ID NOs: 1-8 are most closely related to the following bacterial species: Clostridium bolteae, Anaerotruncus colihominis, Sellimonas intestinalis, Clostridium symbiosum, Blautia producta, Dorea longicatena, Clostridium innocuum, and Flavonifractor plautii (see, e.g., Table 1). Thus, in one aspect it should be appreciated that each row of Table 1, the bacterial strains are highly similar and/or are identical. In some embodiments, in context of the instant disclosure the names of bacterial strains within a row of Table 1 can be used interchangeably.

Bacterial strains of species listed in Table 1 are available to those of ordinary skill in the art, from repositories such as the American Type Culture Collection (ATCC) and Deutsche Sammlung von Mikroorganismen und Zellkulturen (DSMZ). ATCC is a repository for reference cultures and biological materials, including microorganisms, distributing cultures to scientists worldwide. Similarly, DSMZ is an active collection of biological resources including microorganisms, from which catalogued strains are available.

Aspects of the disclosure relate to bacterial strains with 16S rDNA sequences that have homology or identity to a nucleic acid sequence of any one of the sequences of the bacterial strains or species described herein. In some embodiments, the bacterial strain has at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% identity relative to any of the strains or bacterial species described herein over a specified region of nucleic acid or amino acid sequence or over the entire nucleic acid or amino acid sequence. It would be appreciated by one of skill in the art that the terms “homology” or “percent homology,” may be used interchangeably with “identity” or “percent identity,” respectively. In the context of two or more nucleic acid sequences or amino acid sequences, the terms homology or identity refer to a measure of similarity between two or more sequences or portion(s) thereof. The homology or identity may exist over a region of a sequence that is at least about 50 nucleotides in length, or more preferably over a region that is 100 to 500 or 1000 or more nucleotides in length. In some embodiments, the homology or identity exists over the length the 16S rRNA or 16S rDNA sequence, or a portion thereof.

In some embodiments, the compositions include one or more bacterial strains, wherein the one or more bacterial strains comprising 16S rDNA sequences having at least 97% identity with nucleic acid sequences of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the compositions include one or more bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% identity with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

In some embodiments, the compositions consist of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% identity with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

Additionally, or alternatively, two or more sequences may be assessed for the identity between the sequences. The terms “identical” or “percent identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over an entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the compositions include one or more bacterial strains, wherein the one or more bacterial strains comprising 16S rDNA sequences having at least 97% sequence identity with nucleic acid sequences of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the compositions include one or more bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

In some embodiments, the compositions consist of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

Additionally, or alternatively, two or more sequences may be assessed for the alignment between the sequences. The terms “alignment” or “percent alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region of the nucleic acid or amino acid sequence or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology or identity algorithm of Smith and Waterman (1970) Adv. Appl. Math. 2:482c, by the homology or identity alignment algorithm of Needleman and Wunsch, J. Mol. Biol. (1970) 48:443, by the search for similarity method of Pearson and Lipman. Proc. Natl. Acad. Sci. USA (1998) 85:2444, by computerized implementations of these algorithms (GAP, BESTFIT, FASTA, and TFASTA in the Wisconsin Genetics Software Package, Genetics Computer Group. Madison. WI), or by manual alignment and visual inspection (see. E.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402, and Altschul et al., J. Mol. Biol. (1990) 215:403-410, respectively.

It should be appreciated that the terms “bacteria,” “bacterial strain,” and “bacterial strains” as used herein are interchangeable.

In some embodiments of the compositions provided herein, one or more of the bacterial strains are human-derived bacteria, meaning the one or more bacterial strains were obtained from or identified from a human or a sample therefrom (e.g., a human donor). In some embodiments of the compositions provided herein, all of the bacterial strains are human-derived bacteria. In some embodiments of the compositions provided herein, the bacterial strains are derived from more than one human donor.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial strains that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments of the compositions provided herein, the composition includes one or more anaerobic bacteria. In some embodiments of the compositions provided herein, the composition includes only anaerobic bacteria. In some embodiments of the compositions provided herein, the composition includes one or more facultative anaerobic bacteria. In some embodiments of the compositions provided herein, the composition includes only facultative anaerobic bacteria. In some embodiments of the compositions provided herein, the composition includes one or more obligate anaerobic bacteria. In some embodiments of the compositions provided herein, the composition includes only obligate anaerobic bacteria.

In some embodiments of the compositions provided herein, one or more of the bacterial strains is a spore-former. In some embodiments of the compositions provided herein, one or more of the bacterial strains is in spore form. In some embodiments of the compositions provided herein, one or more of the bacterial strains is a non-spore former.

In some embodiments, the compositions described herein comprise spore forming and non-spore forming bacterial strains. In some embodiments, the compositions described herein comprise spore-forming bacterial strains. In some embodiments, the compositions described herein comprise only spore-forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form (as discussed above, spore forming bacteria can also be in vegetative form). In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the compositions provided herein, in some embodiments, the bacterial strains are purified. In any of the compositions provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals. As used herein, the term “isolated” refers to bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

The methods described herein involve administering any of the compositions described herein to a subject that has or has had a C. difficile infection, neutropenia, cardiovascular disease, atherosclerosis, stroke, asthma, or cancer, or is at elevated risk (compared to a baseline risk in the general population) of experiencing neutropenia, cardiovascular disease, atherosclerosis, stroke, asthma, or cancer. As used herein, “subject,” “individual,” and “patient” are used interchangeably, and refer to a vertebrate, preferably a mammal such as a human. Mammals include, but are not limited to, humans, non-human primates or murine, bovine, equine, canine or feline species. In some embodiments, the subject is a human. In some embodiments, the human subject is a neonatal subject, a pediatric subject, an adolescent subject, an adult subject, or a geriatric subject.

In some embodiments, the subject has had a C. difficile infection. In some embodiments, the subject has been treated for a C. difficile infection. In some embodiments, the subject has been administered an antibiotic for treatment of a C. difficile infection. In some embodiments, the antibiotic is selected from vancomycin, kanamycin, gentamicin, colistin, metronidazole, clindamycin, fidaxomicin, penicillin, streptomycin, and cefoperazone.

In some embodiments, the subject has neutropenia. In some embodiments, the subject is at risk of having neutropenia. In some embodiments, the subject is at an elevated risk of having neutropenia, compared to a baseline risk of neutropenia in the general population. Non-limiting examples of risk factors for neutropenia include older age, cancer, use of myelosuppressive chemotherapeutic agents, and use of antibiotics.

In some embodiments, the subject has cardiovascular disease. In some embodiments, the subject is at risk of having cardiovascular disease. In some embodiments, the subject is at an elevated risk of having cardiovascular disease, compared to a baseline risk of cardiovascular disease in the general population. Non-limiting examples of risk factors for cardiovascular disease include high cholesterol, high triglyceride levels, high blood pressure (hypertension), smoking, diabetes, and obesity.

In some embodiments, the subject has atherosclerosis. In some embodiments, the subject is at risk of having atherosclerosis. In some embodiments, the subject is at an elevated risk of having atherosclerosis, compared to a baseline risk of atherosclerosis in the general population. Non-limiting examples of risk factors for atherosclerosis include high cholesterol, high triglyceride levels, high blood pressure (hypertension), smoking, diabetes, and obesity.

In some embodiments, the subject has a stroke. In some embodiments, the subject is at risk of having a stroke. In some embodiments, the subject is at an elevated risk of having a stroke, compared to a baseline risk of a stroke in the general population. Non-limiting examples of risk factors for stroke include high blood pressure (hypertension), high cholesterol, high triglyceride levels, smoking, diabetes, obesity, viral infections, anxiety, stress.

In some embodiments, the subject has asthma. In some embodiments, the subject has nonallergic asthma. In some embodiments, the subject is at risk of having asthma. In some embodiments, the subject is at an elevated risk of having asthma, compared to a baseline risk of asthma in the general population. Non-limiting examples of risk factors for asthma include allergy, obesity, and smoking.

In some embodiments, the subject has cancer. In some embodiments, the subject is at risk of having cancer. In some embodiments, the subject is at an elevated risk of having cancer, compared to a baseline risk of cancer in the general population. Non-limiting examples of risk factors for cancer include older age, smoking, obesity, alcohol use, and radiation exposure.

Any of the compositions described herein may be administered to a subject in a therapeutically effective amount or a dose of a therapeutically effective amount to treat or prevent recurrent C. difficile infection. Any of the compositions described herein may be administered to a subject in a therapeutically effective amount or a dose of a therapeutically effective amount to treat or prevent neutropenia. Any of the compositions described herein may be administered to a subject in a therapeutically effective amount or a dose of a therapeutically effective amount to treat or prevent cardiovascular disease. Any of the compositions described herein may be administered to a subject in a therapeutically effective amount or a dose of a therapeutically effective amount to treat or prevent atherosclerosis. Any of the compositions described herein may be administered to a subject in a therapeutically effective amount or a dose of a therapeutically effective amount to treat or prevent stroke. Any of the compositions described herein may be administered to a subject in a therapeutically effective amount or a dose of a therapeutically effective amount to treat or prevent asthma. Any of the compositions described herein may be administered to a subject in a therapeutically effective amount or a dose of a therapeutically effective amount to treat or prevent cancer.

The terms “treat” or “treatment” refer to reducing or alleviating one or more of the symptoms associated with a disease or disorder (e.g., neutropenia). In some embodiments, the therapeutically effective amount is an amount sufficient to treat recurrent C. difficile infection, neutropenia, cardiovascular disease, atherosclerosis, stroke asthma, or cancer. The terms “prevent” or “prevention” encompass prophylactic administration and may reduce the incidence or likelihood of experiencing a disease or disorder (e.g., neutropenia). In some embodiments, the therapeutically effective amount is an amount sufficient to reduce the incidence or likelihood of experiencing recurrent C. difficile infection, neutropenia, cardiovascular disease, atherosclerosis, stroke asthma, or cancer. In some embodiments, the therapeutically effective amount is an amount sufficient to reduce the incidence or likelihood of experiencing one or more symptoms of recurrent C. difficile infection, neutropenia, cardiovascular disease, atherosclerosis, stroke asthma, or cancer. In some embodiments, the therapeutically effective amount is an amount sufficient to reduce the incidence or likelihood of experiencing severe recurrent C. difficile infection, neutropenia, cardiovascular disease, atherosclerosis, stroke asthma, or cancer. In some embodiments, the therapeutically effective amount is an amount sufficient to reduce the incidence or likelihood of severe recurrent C. difficile infection. In some embodiments, the therapeutically effective amount is an amount sufficient to reduce the incidence or likelihood of a life-threatening recurrent C. difficile infection. In some embodiments, the therapeutically effective amount is an amount sufficient to reduce the incidence or likelihood of a fulminant recurrent C. difficile infection. Severe infections are characterized by elevated white blood cell counts (at least 15,000 cells/mL blood) and reduced kidney function (serum creatinine >1.5 mg/dL). Fulminant infections are characterized by shock, hypotension, ileus, and/or toxic megacolon.

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein. In some embodiments, the therapeutically effective amount is an amount sufficient to treat C. difficile infection. In some embodiments, the therapeutically effective amount is an amount sufficient to treat recurrent C. difficile infection. In some embodiments, the therapeutically effective amount is an amount sufficient to treat neutropenia. In some embodiments, the therapeutically effective amount is an amount sufficient to treat cardiovascular disease. In some embodiments, the therapeutically effective amount is an amount sufficient to treat atherosclerosis. In some embodiments, the therapeutically effective amount is an amount sufficient to treat stroke. In some embodiments, the therapeutically effective amount is an amount sufficient to treat asthma. In some embodiments, the therapeutically effective amount is an amount sufficient to treat cancer. In some embodiments, the therapeutically effective amount is an amount sufficient to induce one or more cytokines in a subject. In some embodiments, the therapeutically effective amount is an amount sufficient to induce amphiregulin production by regulatory T cells in a subject. In some embodiments, the therapeutically effective amount is an amount sufficient to promote tissue repair. It should be appreciated that the term “effective amount,” in reference to a composition comprising bacterial strains, may be expressed as the number of bacteria or colony forming units (CFUs) to be administered. It should further be appreciated that the bacteria can multiply once administered. Thus, administration of even relatively small few bacteria may have therapeutic effects.

In some embodiments, administration of the compositions described herein reduces the risk of recurrent C. difficile infection (rCDI) in a subject. rCDI is CDI that occurs more than once in the same subject. rCDI is associated with reduced abundance of SCFAs, increased abundance of primary bile acids, and reduced abundance of secondary bile acids. Some aspects of the compositions and methods described herein decrease the abundance of primary bile acids, increase the abundance of secondary bile acids, and/or promote conversion of primary bile acids into secondary bile acids. Bile acids are steroid acids that allow the digestion of dietary fats and oils by acting as surfactants that turn the fats and oils into micelles. Bile acids also act as hormones utilizing the farnesoid X receptor and GBPAR1. Primary bile acids are synthesized in the liver from cholesterol and a conjugated with either taurine or glycine prior to secretion. When the primary bile acids are secreted into the lumen of the intestine, bacteria partially dehydroxylate and remove the glycine or taurine groups, forming secondary bile acids.

Non-limiting examples of primary bile acids are cholic acid (CA), chenodeoxycholic acid (CDCA), glycocholic acid (GCA), glycochenodeoxycholic acid (GCDCA), glycodeoxycholic acid (GDCA), taurocholic acid (TCA), and taurochenodeoxycholic acid (TCDCA). Non-limiting examples of secondary bile acids are deoxycholic acid (DCA), lithocholic acid (LCA), ursodeoxycholic acid (UDCA), taurodeoxycholic acid (TDCA), taurolithocholic acid (TLCA), and tauroursodeoxycholic acid (TUDCA).

Non-limiting examples of secondary bile acids are deoxycholic acid (DCA), lithocholic acid (LCA), 3-oxolithocholic acid (3-oxoLCA), taurolithocholic acid (TLCA), taurodeoxycholic aicd (TDCA), glycolithocholic (GLCA), glycodeoxycholic acid (GDCA), ursodeoxycholic acid (UDCA), tauroursodeoxycholic acid (TUDCA), glycoursodeoxycholic acid (GUDCA), and isoallolithocholic acid+isolithocholic acid (Isoallo-+IsoLCA).

Several diseases and disorders, such as inflammatory bowel disease, cancer (e.g., colorectal carcinoma), and pathogenic organism infections (e.g., Clostridium difficile infection), are associated with increased primary bile acids and reduced secondary bile acids. The primary bile acids are reduced, and the secondary bile acids are increased following fecal matter transplant (FMT). See, e.g., Seekatz, et al., Anaerobe (2018) 53: 64-73. In some embodiments, administration of the bacterial strains or a composition as described herein reduces primary bile acids and/or increases secondary bile acids.

In some embodiments, the levels of primary bile acids are reduced by 2-fold to 100,000-fold following administration of the bacterial strains or any of the compositions described herein. In some embodiments, the levels of primary bile acids are reduced by 2-fold to 1,000-fold following administration of the bacterial strains or any of the compositions described herein. In some embodiments, the levels of primary bile acids are reduced by 10-fold to 1,000-fold following administration of the bacterial strains or any of the compositions described herein. In some embodiments, the levels of primary bile acids are reduced 20-fold to 10,000-fold following administration of the bacterial strains or any of the compositions described herein. In some embodiments, the levels of primary bile acids are reduced by 2-fold, 5-fold, 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 40,000-fold, 50,000-fold, 60,000-fold, 70,000-fold, 80,000-fold, 90,000-fold, or 100,000-fold following administration of the bacterial strains or any of the compositions described herein.

In some embodiments, the levels of secondary bile acids are increased by 2-fold to 10,000-fold following administration of the bacterial strains or any of the compositions described herein. In some embodiments, the levels of secondary bile acids are increased by 10-fold to 10,000-fold following administration of the bacterial strains or any of the compositions described herein. In some embodiments, the levels of secondary bile acids are increased by 10-fold to 1,000-fold following administration of the bacterial strains or any of the compositions described herein. In some embodiments, the levels of secondary bile acids are increased by 20-fold to 100-fold following administration of the bacterial strains or any of the compositions described herein. In some embodiments, the levels of secondary bile acids are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 10,000-fold following administration of the bacterial strains or any of the compositions described herein.

Some embodiments of the compositions and methods described herein increase production of regulatory metabolites, such as short chain fatty acids (e.g., in the gastrointestinal tract of the subject), in a subject. In some embodiments, the methods involve administering to a subject one or more compositions containing bacterial strains that produce short chain fatty acids. SCFAs are abundant in healthy subjects and decreased in subjects having particular diseases and disorders.

SCFA produced in the gastrointestinal tract are thought to function as signaling molecules between the gut microbiota and the host organism, with the SCFA playing a role in local, intermediary and peripheral metabolism of the host. See, e.g., Morrison, et al. Gut Microbes (2016) 7(3): 189-200.

Short chain fatty acids (SCFAs) are fatty acids containing six or fewer carbon atoms. They are produced when dietary fiber is fermented in the intestine. They are primarily absorbed in the portal vein following lipid digestion. SCFAs can affect the production of lipids, energy, and vitamins, as well as playing a critical role in maintaining intestinal epithelial cell membrane integrity. Examples of SCFA include, without limitation, formic acid, acetic acid, butyric acid, isobutyric acid, valeric acid, or isovaleric acid. In some embodiments, the SCFA is butyric acid (butyrate). In some embodiments, the SCFA is succinic acid. In some embodiments, the SCFA is valeric acid. In some embodiments, the SCFA is hexanoic acid. In some embodiments, the SCFA is propionic acid. In some embodiments, the SCFA is isovaleric acid. In some embodiments, the SCFA is isobutyric acid. In some embodiments, the SCFA is 2-methylbutyric acid. In some embodiments, the SCFA is acetic acid.

In some embodiments, SCFAs are increased by 2-fold to 10,000-fold following administration of the bacterial strains or any of the compositions described herein. In some embodiments, SCFAs are increased by 10-fold to 500-fold following administration of the bacterial strains or any of the compositions described herein. In some embodiments, SCFAs are increased by 2-fold to 250-fold following administration of the bacterial strains or any of the compositions described herein. In some embodiments, SCFAs are increased by 100-fold to 500-fold following administration of the bacterial strains or any of the compositions described herein. In some embodiments, SCFAs are increased by 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, or 500-fold following administration of the bacterial strains or any of the compositions described herein.

Some aspects of the compositions and methods described herein increase production of regulatory metabolites, such as indoles (e.g., in the gastrointestinal tract of the subject). In some embodiments, the methods involve administering to a subject one or more of the compositions described herein containing bacterial strains that produce indoles. Indoles are abundant in healthy subjects and decreased in subjects having particular diseases and disorders.

Indoles are tryptophan derivatives and are produced when bacteria catabolize tryptophan in the intestine. Indoles produced in the gastrointestinal tract are thought to contribute to intestinal and systemic homeostasis and may hinder the development of hepatic pathologies such as alcoholic steatohepatitis and nonalcoholic fatty liver disease. See, e.g., Hendrikx et al., J Intern Med. (2019) 286(1):32-40. Examples of indoles include, without limitation, kynurenic acid, serotonin, nicotinic acid, indole, indole 3-acetic, and indole 3-propionic acid.

In some embodiments, the levels of indoles are increased by 2-fold to 10,000-fold following administration of the bacterial strains or any of the compositions described herein. In some embodiments, the levels of indoles are increased by 10-fold to 10,000-fold following administration of the bacterial strains or any of the compositions described herein. In some embodiments, the levels of indoles are increased by 10-fold to 1,000-fold following administration of the bacterial strains or any of the compositions described herein. In some embodiments, the levels of indoles are increased by 20-fold to 100-fold following administration of the bacterial strains or any of the compositions described herein. In some embodiments, the levels of indoles are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 10,000-fold following administration of the bacterial strains or any of the compositions described herein.

In some embodiments, the compositions provided herein induce the proliferation and/or accumulation of regulatory T cells in the subject. As will be evident to one of ordinary skill in the art, regulatory T cells, also referred to as “Tregs,” are a subset of T lymphocytes that are generally thought to suppress an abnormal or excessive immune response and play a role in immune tolerance. Regulatory T cells may be identified based expression of the markers Foxp3 and CD4 (Foxp3+CD4+). The term regulatory T cells may also include Foxp3-negative regulatory T cells that are IL-10-producing CD4-positive T cells.

In some embodiments, the compositions provided herein induce the proliferation and/or accumulation of ST2+ Tregs in the subject. ST2, also known as IL-1 receptor-like 1 (IL1RL1) or IL-33 receptor (IL-33R), binds to IL-33, forming a heterodimer with IL-1 receptor accessory protein (ILIRAP). Binding of the ST2/IL1RAP heterodimer to IL-33 leads to intracellular recruitment of MyD88, which promotes FoxP3 and GATA3 expression, while also promoting Treg function and proliferation by enhancing TGF-β1-mediated differentiation though a p38-dependent mechanism. See, e.g., Griesenauer et al., Front Immunol. 2017. 24(8): 475. MyD88 recruitment also recruits IL-1R-associated kinases (IRAK1, IRAK2, IRAK3, IRAK4) and TRAF6, leading to activation of either the NF-1B or AP-1 (via MAPK) pathways. See, e.g., Chang et al., Transl Perioper & Pain Med, 2016. 1(2): 24-32. These signaling events promote a tissue repair phenotype in Tregs, which may promote tissue repair through multiple mechanisms including mediating tolerance to inflammation, negatively regulate potentially damaging inflammatory cells (e.g., neutrophils and macrophages), and produce growth factors and mediators including amphiregulin (AREG), keratinocyte growth factor (KGF), and/or TGF-β. Such Tregs stimulated by IL-33 exert cardioprotective effects, thereby mitigating the effects of atherosclerosis, through multiple mechanisms, such as reducing cell death of cardiomyocytes, expansion of M2 macrophages, a reparative macrophage subset, and inducing production of IL-5, which promotes generation of cardioprotective antioxidized low-density lipoprotein (ox-LDL) antibodies. See, e.g., Chen et al., Cell Physiol Biochem. 2018. 49(1):349-358; Miller et al., J Exp Med. 2008. 205:339-346. IL-33 signaling in neuronal macrophages (microglia) induces a similar protective response, including phagocytosis of extracellular matrix (regulating synapse remodeling), clearance of A3 (mitigating neuroinflammation), and preserving oligodendrocytes and oligodendrocyte precursor cells, thereby mitigating the effects of ischemic injury in stroke. See, e.g., Xie et al., Stroke. 2021. 52(6):2150-2161. Neuroprotective benefits of IL-33 in stroke are also associated with increased abundance of IL-10-producing Tregs. See, e.g., Zhang et al., JCI Insight. 2018. 3(18):e121560.

In some embodiments, the therapeutically effective amount is an amount sufficient to induce the proliferation and/or accumulation of Tregs in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of Tregs in a subject that has not received any of the compositions described herein or as compared to a fecal sample from the same subject that was collected prior to administration of any of the compositions. In some embodiments, the therapeutically effective amount is an amount sufficient to induce the proliferation and/or accumulation of ST2+ Tregs in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of ST2+ Tregs in a subject that has not received any of the compositions described herein or as compared to a fecal sample from the same subject that was collected prior to administration of any of the compositions.

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and ex vivo effects. In some embodiments, the proliferation and/or accumulation of regulatory T cells may be assessed by detecting and/or quantifying the number of cells that express markers of regulatory T cells (e.g., FoxP3 and CD4), for example by flow cytometry. In some embodiments, the proliferation and/or accumulation of regulatory T cells may be assessed by quantifying the number or percentage of FoxP3+CD4+ cells that also express an additional surface protein, such as ST2. In some embodiments, the proliferation and/or accumulation of regulatory T cells may be assessed by quantifying the number or percentage of FoxP3+CD4+ cells that also express a protein associated with a certain phenotype, such as one associated with tissue repair. Non-limiting examples of proteins expressed by Tregs associated with tissue repair include (i) transcription factors or activators of a repair pathway, such as GATA3, PPARG, and/or BATF; (ii) cytokines (e.g., AREG); growth factors (e.g., KGF, TGF-β); and/or (iii) chemokine receptors (e.g., CCR2, CCR5, CCR8).

In some embodiments, the proliferation and/or accumulation of regulatory T cells may be assessed by determining the activity of the regulatory T cells, such as the production of cytokines (e.g., IL-10). In some embodiments, the proliferation and/or accumulation of regulatory T cells may be assessed by determining the production of amphiregulin.

In some embodiments, the therapeutically effective amount is an amount sufficient to induce production of one or more cytokines in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of the cytokine in a subject (e.g., a subject with neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the compositions or methods described herein induce the production of one or more cytokines in the subject. Methods of measuring cytokine production are known in the art, and include, without limitation, ELISA, Luminex®, qRT-PCR, and western blotting. In some embodiments, a composition or method induces 1, 2, 3, 4, 5, 6, 7, 8, 9, 10, 11, 12, 13, 14, 15, 16, 17, 18, 19, or 20 cytokines selected from TSLP, MIP-1α, MCP-3, LIF, IL-9, IL-5, IL-33, IL-28A, IL-23, IL-20, IL-12(p70), IL-10, IFN-γ, fractalkine, eotaxin-3, G-CSF, CXCL9, IL-1α, IL-17F, and TPO, in a subject.

In some embodiments, a composition or method induces calprotectin production in a subject. Calprotectin is the major component of neutrophil cytosol, and so its abundance is indicative of neutrophil presence (e.g., fecal calprotectin is indicative of neutrophil infiltration into the intestinal lumen, and serum calprotectin is indicative of circulating neutrophils). In some embodiments, the composition or method induces the production of TSLP in a subject. In some embodiments, the composition or method induces the production of MIP-1α in a subject. In some embodiments, the composition or method induces the production of MCP-3 in a subject. In some embodiments, the composition or method induces the production of LIF in a subject. In some embodiments, the composition or method induces the production of IL-9 in a subject. In some embodiments, the composition or method induces the production of IL-5 in a subject. In some embodiments, the composition or method induces the production of IL-33 in a subject. In some embodiments, the composition or method induces the production of IL-28A in a subject. In some embodiments, the composition or method induces the production of IL-23 in a subject. In some embodiments, the composition or method induces the production of IL-20 in a subject. In some embodiments, the composition or method induces the production of IL-12(p70) in a subject. In some embodiments, the composition or method induces the production of IL-10 in a subject. In some embodiments, the composition or method induces the production of IFN-γ in a subject. In some embodiments, the composition or method induces the production of fractalkine in a subject. In some embodiments, the composition or method induces the production of eotaxin-3 in a subject. In some embodiments, the composition or method induces the production of G-CSF in a subject. In some embodiments, the composition or method induces the production of CXCL9 in a subject. In some embodiments, the composition or method induces the production of IL-1α in a subject. In some embodiments, the composition or method induces the production of IL-17F in a subject. In some embodiments, the composition or method induces the production of TPO in a subject.

In some embodiments, the therapeutically effective amount is an amount sufficient to induce the production of calprotectin in a subject. In some embodiments, the therapeutically effective amount is an amount sufficient to induce production of calprotectin in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of calprotectin in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of TSLP in a subject. In some embodiments, the therapeutically effective amount is an amount sufficient to induce production of TSLP in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of TSLP in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of MIP-1α in a subject. In some embodiments, the therapeutically effective amount is an amount sufficient to induce production of MIP-1α in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of MIP-1α in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of MCP-3 in a subject. In some embodiments, the therapeutically effective amount is an amount sufficient to induce production of MCP-3 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of MCP-3 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of LIF in a subject. In some embodiments, the therapeutically effective amount is an amount sufficient to induce production of LIF in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of LIF in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-9 in a subject. In some embodiments, the therapeutically effective amount is an amount sufficient to induce production of IL-9 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-9 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-5 in a subject. In some embodiments, the therapeutically effective amount is an amount sufficient to induce production of IL-5 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-5 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-33 in a subject. In some embodiments, the therapeutically effective amount is an amount sufficient to induce production of IL-33 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-33 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-28A in a subject. In some embodiments, the therapeutically effective amount is an amount sufficient to induce production of IL-28A in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-28A in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-23 in a subject. In some embodiments, the therapeutically effective amount is an amount sufficient to induce production of IL-23 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-23 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-20 in a subject. In some embodiments, the therapeutically effective amount is an amount sufficient to induce production of IL-20 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-20 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-12(p70) in a subject. In some embodiments, the therapeutically effective amount is an amount sufficient to induce production of IL-12(p70) in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-12(p70) in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-10 in a subject. In some embodiments, the therapeutically effective amount is an amount sufficient to induce production of IL-10 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-10 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IFN-γ in a subject. In some embodiments, the therapeutically effective amount is an amount sufficient to induce production of IFN-γ in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IFN-γ in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of fractalkine in a subject. In some embodiments, the therapeutically effective amount is an amount sufficient to induce production of fractalkine in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of fractalkine in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of eotaxin-3 in a subject. In some embodiments, the therapeutically effective amount is an amount sufficient to induce production of eotaxin-3 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of eotaxin-3 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of G-CSF in a subject. In some embodiments, the therapeutically effective amount is an amount sufficient to induce production of G-CSF in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of G-CSF in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of CXCL9 in a subject. In some embodiments, the therapeutically effective amount is an amount sufficient to induce production of CXCL9 in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of CXCL9 in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-1α in a subject. In some embodiments, the therapeutically effective amount is an amount sufficient to induce production of IL-1α in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-1α in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of IL-17F in a subject. In some embodiments, the therapeutically effective amount is an amount sufficient to induce production of IL-17F in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IL-17F in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

In some embodiments, the therapeutically effective amount is an amount sufficient to induce the production of TPO in a subject. In some embodiments, the therapeutically effective amount is an amount sufficient to induce production of TPO in the subject (or in a sample obtained from the subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of TPO in a subject (e.g., a subject with a C. difficile infection, neutropenia, cardiovascular disease, atherosclerosis, stroke, asthma, or cancer) that has not received any of the compositions as described herein, or as compared to a blood or other sample from the same subject that was collected prior to administration of any of the compositions.

As described herein, neutropenia is a condition characterized by a low abundance of circulating neutrophils, below the standard count of 1,500 cells per microliter. The causes of neutropenia are thought to include, for example, use of drugs including quinidine, aminopyrine, cephalosporin, sulfonamides, hydralazine, penicillins, heavy metals, or phenothiazine. Other conditions, including rheumatoid arthritis, inflammatory bowel disease, chronic autoimmune hepatitis, granulomatosis, Hodgkin's lymphoma, and Sjogren's syndrome, are associated with neutropenia. Neutropenia is also observed in subjects with folate, vitamin B12, and/or copper deficiency. The resulting neutrophil deficiency increases the risk of infections, as well as complications from infections, such as dissemination of bacteria or viruses to other anatomical sites, bacteremia or viremia, sepsis, and death. See, e.g., Justiz Vaillant A A, Zito P M. Neutropenia. [Updated 2022 Aug. 25]. In: StatPearls [Internet]. Treasure Island (FL): StatPearls Publishing; 2023 January-. NCBI Books NBK507702. As described herein, atherosclerosis is a condition characterized by buildup of plaque inside arteries. These plaques typically comprise lipids that induce an inflammatory response, causing turbulent flow within arteries and cardiovascular disease. Causes of atherosclerosis are thought to include hypercholesterolemia (elevated levels of low-density lipoprotein (LDL)-cholesterol), hypertension, diabetes, smoking, and old age. See, e.g., Pahwa R, Jialal I. Atherosclerosis. [Updated 2022 Aug. 8]. In: StatPearls [Internet]. Treasure Island (FL): StatPearls Publishing; 2023 January-. NCBI Books NBK507799.

As described herein, stroke is a condition characterized by blockage of blood flow to the brain (ischemic stroke) or leakage of blood into the intracranial cavity (hemorrhagic stroke). Many pathologies are associated with ischemic stroke, but leading causes include large vessel disease, small vessel disease, and cardioembolism. Hemorrhagic stroke is typically caused by rupture of small arteries, hypertensive vasculopathy, cerebral amyloid angiopathy, and coagulopathy. See, e.g., Tadi P, Lui F. Acute Stroke. [Updated 2023 Feb. 28]. In: StatPearls [Internet]. Treasure Island (FL): StatPearls Publishing; 2023 January-. NCBI Books NBK535369.

As described herein, asthma is a condition characterized by inflammation and narrowing of the airways. Asthma symptoms include shortness of breath, coughing, and wheezing. The causes of asthma are thought to include a combination of genetic (such as family history of asthma or allergies) and environmental (such as exposure to smoke and particulate matter) factors. Asthma is characterized by acute inflammation, which often follows exposure to an environmental stimulus (e.g., an irritant or allergen). Such inflammation leads to inflammatory cell infiltration into the airways, mucus secretion into the airways, and smooth muscle contraction, with consequent airway obstruction. Over time, such symptoms may become irreversible due to basement membrane thickening, collagen deposition, and airway remodeling. See, e.g., Hashmi M F, Tariq M, Cataletto M E. Asthma. [Updated 2023 Feb. 19]. In: StatPearls [Internet]. Treasure Island (FL): StatPearls Publishing; 2023 January. NCBI Books NBK430901.

As described herein, cancer is a condition characterized by aberrant cellular proliferation. Uncontrolled cell division may lead to formation of tumors in a subject. As tumors grow, they may inhibit blood flow through and function of organs in which they are present, disrupting normal physiology. Furthermore, cancer cells may spread to lymph nodes or other tissues, establishing secondary sites of tumor growth. See, e.g., Castaneda et al., Semin Cancer Biol. 2022. 87:17-31.

Any of the methods described herein may be for the treatment of neutropenia in a subject. As used herein, methods of treating neutropenia involve relieving or alleviating at least one symptom associated with neutropenia, or slowing or reversing the neutropenia progression. In some embodiments, a composition or method induces neutrophil differentiation in a subject. Neutrophil differentiation may be measured by any suitable method, such as flow cytometry analysis of blood samples and evaluation of neutrophil populations as compared to neutrophil precursor populations.

In some embodiments, a composition or method increases the abundance of circulating neutrophils in a subject. Circulating neutrophil abundance may be measured by any suitable method, such as flow cytometry analysis of blood samples.

In some embodiments, a composition or method inhibits infiltration of neutrophils into an intestinal lumen of the subject. Neutrophil infiltration into the intestinal lumen may be evaluated by any suitable method, such as flow cytometry analysis of fecal samples, flow cytometry analysis of intestinal biopsy samples, and/or analysis of calprotectin levels in fecal samples.

Any of the methods described herein may be for the treatment of cardiovascular disease in a subject. As used herein, methods of treating cardiovascular disease involve relieving or alleviating at least one symptom associated with cardiovascular disease, or slowing or reversing the cardiovascular disease progression.

Any of the methods described herein may be for the treatment of atherosclerosis in a subject. As used herein, methods of treating atherosclerosis involve relieving or alleviating at least one symptom associated with atherosclerosis, or slowing or reversing the atherosclerosis progression.

Any of the methods described herein may be for the treatment of atherosclerosis in a subject. As used herein, methods of treating stroke involve relieving or alleviating at least one symptom associated with stroke, or slowing or reversing the stroke progression.

Any of the methods described herein may be for the treatment of asthma in a subject. As used herein, methods of treating asthma involve relieving or alleviating at least one symptom associated with asthma, or slowing or reversing the asthma progression.

Any of the methods described herein may be for the treatment of cancer in a subject. As used herein, methods of treating cancer involve relieving or alleviating at least one symptom associated with cancer, or slowing or reversing the cancer progression.

In some embodiments, the subject has a C. difficile infection.

In some embodiments, the subject does not have a C. difficile infection. In some embodiments, the subject has not had a C. difficile infection.

In some embodiments, the bacterial strains of the compositions provided herein can treat and/or prevent recurrent C. difficile infection due to synergy between the bacterial strains. In some embodiments, the bacterial strains of the compositions provided herein can treat and/or prevent neutropenia due to synergy between the bacterial strains. In some embodiments, the bacterial strains of the compositions provided herein can treat and/or prevent cardiovascular disease due to synergy between the bacterial strains. In some embodiments, the bacterial strains of the compositions provided herein can treat and/or prevent atherosclerosis due to synergy between the bacterial strains. In some embodiments, the bacterial strains of the compositions provided herein can treat and/or prevent stroke due to synergy between the bacterial strains. In some embodiments, the bacterial strains of the compositions provided herein can treat and/or prevent asthma due to synergy between the bacterial strains. In some embodiments, the bacterial strains of the compositions provided herein can treat and/or prevent cancer due to synergy between the bacterial strains.

As described herein, any of the compositions described herein may be administered to a subject in one dose or in multiple doses (e.g., initial administration), which may be followed by one or more additional doses of any of the compositions described herein. In some embodiments, any of composition described herein may be administered to a subject in one dose or in multiple doses in an initial administration, followed by one or more additional doses of a composition comprising the same one or more bacterial strains as the composition of the initial administration. In some embodiments, any of composition described herein may be administered to a subject in one dose or in multiple doses in an initial administration, followed by one or more additional doses of a composition comprising more total bacteria (colony-forming units) relative to the initial administration of the composition. In some embodiments, any of composition described herein may be administered to a subject in one dose or in multiple doses in an initial administration, followed by one or more additional doses of a composition comprising fewer total bacteria (colony-forming units) relative to the initial administration of the composition. In some embodiments, the initial administration includes at least 1, 2, 3, 4, 5, 6, 7, 8, 9, 10, 11, 12, 13, 14, 15 or more doses of any of the compositions described herein. In some embodiments, the additional administration includes at least 1, 2, 3, 4, 5, 6, 7, 8, 9, 10, 11, 12, 13, 14, 15 or more doses of any of the compositions described herein. In some embodiments, the initial administration comprises two doses of any of the composition and the additional administration comprises three doses of any of the compositions described herein.

In some embodiments, the subject has not received a dose of an antibiotic prior to administration of the composition. In some embodiments, the subject has not been administered an antibiotic at least 1, at least 2, at least 3, at least 5, at least 10, at least 15, at least 20, at least 25, at least 30, at least 60, at least 90, at least 120, at least 180 or at least 360 days prior to administration of the compositions provided herein. In some embodiments, the subject is treated with an amount of antibiotics sufficient to allow for the grafting of the one or more strains of the compositions provided herein.

In some embodiments, the dosing regimen entails administration of multiple doses of any of the compositions described herein. In some embodiments, the composition is administered orally to the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered to the subject in multiple doses at a regular interval, such as every day, every 2 days, every 3 days, every 4 days, every 5 days, every 6 days, every week, every 2 weeks, every 4 weeks, every month, every 2 months, every 3 months, every 4 months, every 5 months, every 6 months, or more. In some embodiments, one dose of any of the compositions described herein is administered and a second dose of the composition is administered the following day (e.g., consecutive days). In some embodiments, one dose of any of the compositions described herein is administered and each of the additional doses of the composition are administered on consecutive days (e.g., first dose on day 1, second dose of day 2, third dose on day 3, etc.). In some embodiments, multiple doses are administered over multiple days. In some embodiments, the subject is administered at least 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹, 5.1×10⁹, 5.2×10⁹, 5.3×10⁹, 5.4×10⁹, 5.5×10⁹, 5.6×10⁹, 5.7×10⁹, 5.8×10⁹, 5.9×10⁹, 6.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 7.1×10⁹, 7.2×10⁹, 7.3×10⁹, 7.4×10⁹, 7.5×10⁹, 7.6×10⁹, 7.7×10⁹, 7.8×10⁹, 7.9×10⁹, 8.0×10⁹, 8.1×10⁹, 8.2×10⁹, 8.3×10⁹, 8.4×10⁹, 8.5×10⁹, 8.6×10⁹, 8.7×10⁹, 8.8×10⁹, 8.9×10⁹, 9.0×10⁹, 9.1×10⁹, 9.2×10⁹, 9.3×10⁹, 9.4×10⁹, 9.5×10⁹, 9.6×10⁹, 9.7×10⁹, 9.8×10⁹, or 9.9×10⁹ colony forming units of bacteria per day. In some embodiments, the subject is administered at least 1.0×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2.0×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3.0×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4.0×10¹⁰, 4.1×10¹⁰, 4.2×10¹⁰, 4.3×10¹⁰, 4.4×10¹⁰, 4.5×10¹⁰, 4.6×10¹⁰, 4.7×10¹⁰, 4.8×10¹⁰, 4.9×10¹⁰, 5.0×10¹⁰, 5.1×10¹⁰, 5.2×10¹⁰, 5.3×10¹⁰, 5.4×10¹⁰, 5.5×10¹⁰, 5.6×10¹⁰, 5.7×10¹⁰, 5.8×10¹⁰, 5.9×10¹⁰, 6.0×10¹⁰, 6.1×10¹⁰, 6.2×10¹⁰, 6.3×10¹⁰, 6.4×10¹⁰, 6.5×10¹⁰, 6.6×10¹⁰, 6.7×10¹⁰, 6.8×10¹⁰, 6.9×10¹⁰, 7.0×10¹⁰, 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, 7.9×10¹⁰, 8.0×10¹⁰, 8.1×10¹⁰, 8.2×10¹⁰, 8.3×10¹⁰, 8.4×10¹⁰, 8.5×10¹⁰, 8.6×10¹⁰, 8.7×10¹⁰, 8.8×10¹⁰, 8.9×10¹⁰, 9.0×10¹⁰, 9.1×10¹⁰, 9.2×10¹⁰, 9.3×10¹⁰, 9.4×10¹⁰, 9.5×10¹⁰, 9.6×10¹⁰, 9.7×10¹⁰, 9.8×10¹⁰, or 9.9×10¹⁰ colony forming units of bacteria in total. In some embodiments, the subject is administered at least 1.0×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2.0×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3.0×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4.0×10¹¹, 4.1×10¹¹, 4.2×10¹¹, 4.3×10¹¹, 4.4×10¹¹, 4.5×10¹¹, 4.6×10¹¹, 4.7×10¹¹, 4.8×10¹¹, 4.9×10¹¹, 5.0×10¹¹, 5.1×10¹¹, 5.2×10¹¹, 5.3×10¹¹, 5.4×10¹¹, 5.5×10¹¹, 5.6×10¹¹, 5.7×10¹¹, 5.8×10¹¹, 5.9×10¹¹, 6.0×10¹¹, 6.1×10¹¹, 6.2×10¹¹, 6.3×10¹¹, 6.4×10¹¹, 6.5×10¹¹, 6.6×10¹¹, 6.7×10¹¹, 6.8×10¹¹, 6.9×10¹¹, 7.0×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8.0×10¹¹, 8.1×10¹¹, 8.2×10¹¹, 8.3×10¹¹, 8.4×10¹¹, 8.5×10¹¹, 8.6×10¹¹, 8.7×10¹¹, 8.8×10¹¹, 8.9×10¹¹, 9.0×10¹¹, 9.1×10¹¹, 9.2×10¹¹, 9.3×10¹¹, 9.4×10¹¹, 9.5×10¹¹, 9.6×10¹¹, 9.7×10¹¹, 9.8×10¹¹, or 9.9×10¹⁰ colony forming units of bacteria in total.

In some embodiments, the subject is administered a single dose of an antibiotic prior to the administration of any of the compositions described herein. In some embodiments, the subject is administered multiple doses of an antibiotic prior to the administration of any of the compositions described herein. In some embodiments, the subject is administered at least 2, 3, 4, 5 or more doses of an antibiotic prior to the administration of any of the compositions described herein. In some embodiments, the subject is administered a dose of an antibiotic at substantially the same time as the administration of any of the compositions described herein. Examples of antibiotics that can be administered include, without limitation, vancomycin, kanamycin, gentamicin, colistin, metronidazole, clindamycin, fidaxomicin, penicillin, streptomycin, and cefoperazone.

In some embodiments in any one the methods provided herein, a subject is evaluated for the presence of one or more of the bacterial strains of the compositions described herein in the microbiome. In some embodiments, if the subject does not have, or only has a low level of one or more of the bacterial strains of the compositions described herein in the microbiome, any one of the compositions provided herein, or one or more further doses of any one of the compositions provided herein, may be administered.

In some embodiments, the subject is evaluated for the presence of and/or abundance of one or more bacterial strains of the compositions described herein in the microbiome. In some embodiments, if one or more bacterial strains of the compositions are detected at a level above a threshold level, no further compositions or food products containing the bacterial strains are administered to the subject. In some embodiments, if one or more bacterial strains of the compositions colonize the subject to a level above a threshold level, no further compositions or food products containing the bacterial strains are administered to the subject.

The bacterial strains of the composition can be manufactured using fermentation techniques well known in the art. In some embodiments, the active ingredients are manufactured using anaerobic fermenters, which can support the rapid growth of anaerobic bacterial strains. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration and can optionally be dried and lyophilized by techniques well known in the art.

In some embodiments, the live bacterial product may be formulated for administration as a pharmaceutical composition. The term “pharmaceutical composition” as used herein means a product that results from the mixing or combining of at least one active ingredient, such as any of the bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the compositions comprising bacterial strains are formulated for oral delivery. In some embodiments, the bacteria are formulated for delivery to the intestines (e.g., the small intestine and/or the colon). In some embodiments, the bacteria are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See, e.g., Zhang, AAPS PharmSciTech (2016) 17 (1), 56-67).

The compositions comprising bacteria may also be formulated for rectal delivery to the intestine (e.g., the colon). Thus, in some embodiments, the compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH-sensitive compositions, more specifically, buffered sachet formulations or enteric polymers that release their contents when the pH becomes alkaline after the enteric polymers pass through the stomach. When a pH-sensitive composition is used for formulating the pharmaceutical preparation, the pH-sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum) has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. See, e.g., Villena et al., Int J Pharm (2015) 487 (1-2): 314-9.

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, a pharmaceutical composition may include an additional component that enables efficient delivery of the compositions of the disclosure to a desired site, such as the gastrointestinal tract (e.g., the colon).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more bacteria. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more bacteria per milliliter. It should be appreciated that some of the bacteria may not be viable. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of bacteria. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of bacteria per milliliter.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units per milliliter.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria per milliliter.

In some embodiments, the composition includes between 10¹⁰ and 10¹², inclusive, bacterial cells or colony forming units of bacteria per milliliter.

In some embodiments, if a composition includes more than one bacterial strain, each bacterial strain is present in the composition in the same quantities, in terms of bacterial cells or CFUs. For example, a composition comprising Clostridium symbiosum and Blautia producta may comprise about 1×10⁸ CFU of Clostridium symbiosum and 1×10⁸ CFU of Blautia producta per milliliter, or may comprise 1×10⁸ Clostridium symbiosum cells and 1×10⁸ Blautia producta cells per milliliter.

In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each bacterial strain. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each bacterial strain per milliliter. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of each bacterial strain. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of each of the bacterial strains per milliliter.

In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or colony forming units of each bacterial strain. In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain per milliliter.

In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain per milliliter.

In some embodiments, the composition includes between 1×10⁹ and 1×10¹¹, inclusive, bacterial cells or colony forming units of each bacterial strain per milliliter. In some embodiments, the composition includes between 2×10⁹ and 2×10¹⁰, inclusive, bacterial cells or colony forming units of each bacterial strain per milliliter.

Aspects of the present disclosure provide food products comprising any of the compositions provided herein and a nutrient. Also within the scope of the present disclosure are food products comprising any of the bacterial strains described herein and a nutrient. Food products are, in general, intended for the consumption by a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

Food products containing bacterial strains described herein may be produced using methods known in the art and may contain the same amount of bacteria (e.g., by weight, amount or CFU) as the compositions (e.g., pharmaceutical compositions) provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

TABLE 1
Examples of bacterial species of the compositions disclosed herein.

| | SEQ ID | Closest species | | | | |
|---|---|---|---|---|---|---|
| | NO. of | based on Consensus | Closest species | Closest species | | |
| | 16S | SEQ ID # of 16S | based on Sanger | based on WGS | Additional | |
| Strain | rDNA | region as compared | sequencing | compared versus | closely related | Clostridium |
| number | sequence | with 16S database | of 16S region | WG databases | sequences | cluster |
| 1 | 1 | Clostridium bolteae | Clostridium bolteae | Clostridium bolteae | Lachnoclostridium | XIVa |
| | | | | 90A9 | sp.; Enterocloster | |
| | | | | | bolteae | |
| 2 | 2 | Anaerotruncus | Anaerotruncus | Anaerotruncus | | IV |
| | | colihominis | colihominis | colihominis DSM 17241 | | |
| 3 | 3 | Sellimonas | Sellimonas | Sellimonas | Ruminococcus | XIVa |
| | | intestinalis | intestinalis | intestinalis | torques; | |
| | | | | | Eubacterium | |
| | | | | | fissicatena; | |
| | | | | | Drancourtella | |
| | | | | | massiliensis | |
| 4 | 4 | Clostridium symbiosum | Clostridium symbiosum | Clostridium symbiosum | | XIVa |
| | | | | WAL-14163 | | |
| 5 | 5 | Blautia producta | Blautia producta | Clostridium bacterium | Blautia producta | XIVa |
| | | | | UC5.1-1D4 | ATCC 27340; | |
| | | | | | Blautia hominis | |
| 6 | 6 | Dorea longicatena | Dorea longicatena | Dorea longicatena | | XIVa |
| | | | | CAG:42 | | |
| 7 | 7 | Clostridium innocuum | Clostridium innocuum | Erysipelotrichaceae | | XVII |
| | | | | bacterium 21_3 | | |
| 8 | 8 | Flavonifractor plautii | Flavonifractor plautii | Clostridium | Subdolinogranulum | IV |
| | | | | orbiscindens | spp. | |
| | | | | 1_3_50AFAA | | |

### EXEMPLARY SEQUENCES

Strain 1
Clostridium bolteae 16S ribosomal RNA coding sequence (16S rDNA)
(SEQ ID NO: 1)
ATGAGAGTTTGATCCTGGCTCAGGATGAACGCTGGCGGCGTGCCTAACACATGCAAGTCGAACGAAGCAATTAAA
ATGAAGTTTTCGGATGGATTTTTGATTGACTGAGTGGCGGACGGGTGAGTAACGCGTGGATAACCTGCCTCACAC
TGGGGGATAACAGTTAGAAATGACTGCTAATACCGCATAAGCGCACAGTACCGCATGGTACGGTGTGAAAAACTC
CGGTGGTGTGAGATGGATCCGCGTCTGATTAGCCAGTTGGCGGGGTAACGGCCCACCAAAGCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGCGAAAGCCTGATGCAGCGACGCCGCGTGAGTGAAGAAGTATTTCGGTATGTAAAGCTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGCGAAGCAAGTCTGAAGTGAAAACCCAG
GGCTCAACCCTGGGACTGCTTTGGAAACTGTTTTGCTAGAGTGTCGGAGAGGTAAGTGGAATTCCTAGTGTAGCG
GTGAAATGCGTAGATATTAGGAGGAACACCAGTGGCGAAGGCGGCTTACTGGACGATAACTGACGTTGAGGCTCG
AAAGCGTGGGGAGCAAACAGGATTAGATACCCTGGTAGTCCACGCCGTAAACGATGAATGCTAGGTGTTGGGGGG
CAAAGCCCTTCGGTGCCGTCGCAAACGCAGTAAGCATTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCAAGTC
TTGACATCCTCTTGACCGGCGTGTAACGGCGCCTTCCCTTCGGGGCAGGAGAGACAGGTGGTGCATGGTTGTCGT
CAGCTCGTGTCGTGAGATGTTGGGTTAAGTCCCGCAACGAGCGCAACCCTTATCCTTAGTAGCCAGCAGGTAGAG
CTGGGCACTCTAGGGAGACTGCCAGGGATAACCTGGAGGAAGGTGGGGATGACGTCAAATCATCATGCCCCTTAT
GATTTGGGCTACACACGTGCTACAATGGCGTAAACAAAGGGAAGCAAGACAGTGATGTGGAGCAAATCCCAAAAA
TAACGTCCCAGTTCGGACTGTAGTCTGCAACCCGACTACACGAAGCTGGAATCGCTAGTAATCGCGAATCAGAAT
GTCGCGGTGAATACGTTCCCGGGTCTTGTACACACCGCCCGTCACACCATGGGAGTCAGCAACGCCCGAAGTCAG
TGACCCAACTCGCAAGAGAGGGAGCTGCCGAAGGCGGGGCAGGTAACTGGGGTGAAGTCGTAACAAGGTAGCCGT
ATCGGAAGGTGCGGCTGGATCACCTCCTTT
Strain 2
Anaerotruncus colihominis 16S ribosomal RNA coding sequence (16S rDNA)
(SEQ ID NO: 2)
CAAAGAGTTTGATCCTGGCTCAGGACGAACGCTGGCGGCGCGCCTAACACATGCAAGTCGAACGGAGCTTACGTT
TTGAAGTTTTCGGATGGATGAATGTAAGCTTAGTGGCGGACGGGTGAGTAACACGTGAGCAACCTGCCTTTCAGA
GGGGGATAACAGCCGGAAACGGCTGCTAATACCGCATGATGTTGCGGGGGCACATGCCCCTGCAACCAAAGGAGC
AATCCGCTGAAAGATGGGCTCGCGTCCGATTAGCCAGTTGGCGGGGTAACGGCCCACCAAAGCGACGATCGGTAG
CCGGACTGAGAGGTTGAACGGCCACATTGGGACTGAGACACGGCCCAGACTCCTACGGGAGGCAGCAGTGGGGGA
TATTGCACAATGGGCGAAAGCCTGATGCAGCGACGCCGCGTGAGGGAAGACGGTCTTCGGATTGTAAACCTCTGT
CTTTGGGGAAGAAAATGACGGTACCCAAAGAGGAAGCTCCGGCTAACTACGTGCCAGCAGCCGCGGTAATACGTA
GGGAGCAAGCGTTGTCCGGAATTACTGGGTGTAAAGGGAGCGTAGGCGGGATGGCAAGTAGAATGTTAAATCCAT
CGGCTCAACCGGTGGCTGCGTTCTAAACTGCCGTTCTTGAGTGAAGTAGAGGCAGGCGGAATTCCTAGTGTAGCG
GTGAAATGCGTAGATATTAGGAGGAACACCAGTGGCGAAGGCGGCCTGCTGGGCTTTAACTGACGCTGAGGCTCG
AAAGCGTGGGGAGCAAACAGGATTAGATACCCTGGTAGTCCACGCCGTAAACGATGATTACTAGGTGTGGGGGGA
CTGACCCCTTCCGTGCCGCAGTTAACACAATAAGTAATCCACCTGGGGAGTACGGCCGCAAGGTTGAAACTCAAA
GGAATTGACGGGGGCCCGCACAAGCAGTGGAGTATGTGGTTTAATTCGAAGCAACGCGAAGAACCTTACCAGGTC
TTGACATCGGATGCATAGCCTAGAGATAGGTGAAGCCCTTCGGGGCATCCAGACAGGTGGTGCATGGTTGTCGTC
AGCTCGTGTCGTGAGATGTTGGGTTAAGTCCCGCAACGAGCGCAACCCTTATTATTAGTTGCTACGCAAGAGCAC
TCTAATGAGACTGCCGTTGACAAAACGGAGGAAGGTGGGGATGACGTCAAATCATCATGCCCCTTATGACCTGGG
CTACACACGTACTACAATGGCACTAAAACAGAGGGCGGCGACACCGCGAGGTGAAGCGAATCCCGAAAAAGTGTC
TCAGTTCAGATTGCAGGCTGCAACCCGCCTGCATGAAGTCGGAATTGCTAGTAATCGCGGATCAGCATGCCGCGG
TGAATACGTTCCCGGGCCTTGTACACACCGCCCGTCACACCATGGGAGTCGGTAACACCCGAAGCCAGTAGCCTA
ACCGCAAGGGGGGCGCTGTCGAAGGTGGGATTGATGACTGGGGTGAAGTCGTAACAAGGTAGCCGTATCGGAAGG
TGCGGCTGGATCACCTCCTTT
Strain 3
Sellimonas intestinalis 16S ribosomal RNA coding sequence (16S rDNA)
(SEQ ID NO: 3)
ACGAGAGTTTGATCCTGGCTCAGGATGAACGCTGGCGGCGTGCCTAACACATGCAAGTCGAGCGAAGCGCTGTTT
TCAGAATCTTCGGAGGAAGAGGACAGTGACTGAGCGGCGGACGGGTGAGTAACGCGTGGGCAACCTGCCTCATAC
AGGGGGATAACAGTTAGAAATGACTGCTAATACCGCATAAGCGCACAGGACCGCATGGTGTAGTGTGAAAAACTC
CGGTGGTATGAGATGGACCCGCGTCTGATTAGGTAGTTGGTGGGGTAAAGGCCTACCAAGCCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGGGAAACCCTGATGCAGCGACGCCGCGTGAAGGAAGAAGTATTTCGGTATGTAAACTTCTATC
AGCAGGGAAGAAGATGACGGTACCTGAGTAAGAAGCACCGGCTAAATACGTGCCAGCAGCCGCGGTAATACGTAT
GGTGCAAGCGTTATCCGGATTTACTGGGTGTAAAGGGAGCGTAGACGGATAGGCAAGTCTGGAGTGAAAACCCAG
GGCTCAACCCTGGGACTGCTTTGGAAACTGCAGATCTGGAGTGCCGGAGAGGTAAGCGGAATTCCTAGTGTAGCG
GTGAAATGCGTAGATATTAGGAGGAACACCAGTGGCGAAGGCGGCTTACTGGACGGTGACTGACGTTGAGGCTCG
AAAGCGTGGGGAGCAAACAGGATTAGATACCCTGGTAGTCCACGCCGTAAACGATGACTACTAGGTGTCGGTGTG
CAAAGCACATCGGTGCCGCAGCAAACGCAATAAGTAGTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCTGGTC
TTGACATCCGGATGACGGGCGAGTAATGTCGCCGTCCCTTCGGGGCATCCGAGACAGGTGGTGCATGGTTGTCGT
CAGCTCGTGTCGTGAGATGTTGGGTTAAGTCCCGCAACGAGCGCAACCCTTATCTTCAGTAGCCAGCATATAAGG
TGGGCACTCTGGAGAGACTGCCAGGGAGAACCTGGAGGAAGGTGGGGATGACGTCAAATCATCATGCCCCTTATG
GCCAGGGCTACACACGTGCTACAATGGCGTAAACAAAGGGAAGCGAGAGGGTGACCTGGAGCGAATCCCAAAAAT
AACGTCTCAGTTCGGATTGTAGTCTGCAACTCGACTACATGAAGCTGGAATCGCTAGTAATCGCGGATCAGCATG
CCGCGGTGAATACGTTCCCGGGTCTTGTACACACCGCCCGTCACACCATGGGAGTCAGTAACGCCCGAAGCCAGT
GACCCAACCTTAGAGGAGGGAGCTGTCGAAGGCGGGACGGATAACTGGGGTGAAGTCGTAACAAGGTAGCCGTAT
CGGAAGGTGCGGCTGGATCACCTCCTTT
Strain 4
Clostridium symbiosum 16S ribosomal RNA coding sequence (16S rDNA)
(SEQ ID NO: 4)
ATGAGAGTTTGATCCTGGCTCAGGATGAACGCTGGCGGCGTGCCTAACACATGCAAGTCGAACGAAGCGATTTAA
CGGAAGTTTTCGGATGGAAGTTGGATTGACTGAGTGGCGGACGGGTGAGTAACGCGTGGGTAACCTGCCTTGTAC
TGGGGGACAACAGTTAGAAATGACTGCTAATACCGCATAAGCGCACAGTATCGCATGATACAGTGTGAAAAACTC
CGGTGGTACAAGATGGACCCGCGTCTGATTAGCTAGTTGGTAAGGTAACGGCTTACCAAGGCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGCGAAAGCCTGATGCAGCGACGCCGCGTGAGTGAAGAAGTATTTCGGTATGTAAAGCTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGTAAAGCAAGTCTGAAGTGAAAGCCCGC
GGCTCAACTGCGGGACTGCTTTGGAAACTGTTTAACTGGAGTGTCGGAGAGGTAAGTGGAATTCCTAGTGTAGCG
GTGAAATGCGTAGATATTAGGAGGAACACCAGTGGCGAAGGCGACTTACTGGACGATAACTGACGTTGAGGCTCG
AAAGCGTGGGGAGCAAACAGGATTAGATACCCTGGTAGTCCACGCCGTAAACGATGAATACTAGGTGTTGGGGAG
CAAAGCTCTTCGGTGCCGTCGCAAACGCAGTAAGTATTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCAGGTC
TTGACATCGATCCGACGGGGGAGTAACGTCCCCTTCCCTTCGGGGCGGAGAAGACAGGTGGTGCATGGTTGTCGT
CAGCTCGTGTCGTGAGATGTTGGGTTAAGTCCCGCAACGAGCGCAACCCTTATTCTAAGTAGCCAGCGGTTCGGC
CGGGAACTCTTGGGAGACTGCCAGGGATAACCTGGAGGAAGGTGGGGATGACGTCAAATCATCATGCCCCTTATG
ATCTGGGCTACACACGTGCTACAATGGCGTAAACAAAGAGAAGCAAGACCGCGAGGTGGAGCAAATCTCAAAAAT
AACGTCTCAGTTCGGACTGCAGGCTGCAACTCGCCTGCACGAAGCTGGAATCGCTAGTAATCGCGAATCAGAATG
TCGCGGTGAATACGTTCCCGGGTCTTGTACACACCGCCCGTCACACCATGGGAGTCAGTAACGCCCGAAGTCAGT
GACCCAACCGCAAGGAGGGAGCTGCCGAAGGCGGGACCGATAACTGGGGTGAAGTCGTAACAAGGTAGCCGTATC
GGAAGGTGCGGCTGGATCACCTCCTTT
Strain 5
Blautia producta 16S ribosomal RNA coding sequence (16S rDNA)
(SEQ ID NO: 5)
TCAGAGAGTTTGATCCTGGCTCAGGATGAACGCTGGCGGCGTGCTTAACACATGCAAGTCGAGCGAAGCACTTAA
GTGGATCTCTTCGGATTGAAACTTATTTGACTGAGCGGCGGACGGGTGAGTAACGCGTGGGTAACCTGCCTCATA
CAGGGGGATAACAGTTAGAAATGGCTGCTAATACCGCATAAGCGCACAGGACCGCATGGTCTGGTGTGAAAAACT
CCGGTGGTATGAGATGGACCCGCGTCTGATTAGCTAGTTGGAGGGGTAACGGCCCACCAAGGCGACGATCAGTAG
CCGGCCTGAGAGGGTGAACGGCCACATTGGGACTGAGACACGGCCCAGACTCCTACGGGAGGCAGCAGTGGGGAA
TATTGCACAATGGGGGAAACCCTGATGCAGCGACGCCGCGTGAAGGAAGAAGTATCTCGGTATGTAAACTTCTAT
CAGCAGGGAAGAAAATGACGGTACCTGACTAAGAAGCCCCGGCTAACTACGTGCCAGCAGCCGCGGTAATACGTA
GGGGGCAAGCGTTATCCGGATTTACTGGGTGTAAAGGGAGCGTAGACGGAAGAGCAAGTCTGATGTGAAAGGCTG
GGGCTTAACCCCAGGACTGCATTGGAAACTGTTTTTCTAGAGTGCCGGAGAGGTAAGCGGAATTCCTAGTGTAGC
GGTGAAATGCGTAGATATTAGGAGGAACATCAGTGGCGAAGGCGGCTTACTGGACGGTAACTGACGTTGAGGCTC
GAAAGCGTGGGGAGCAAACAGGATTAGATACCCTGGTAGTCCACGCCGTAAACGATGAATACTAGGTGTCGGGTG
GCAAAGCCATTCGGTGCCGCAGCAAACGCAATAAGTATTCCACCTGGGGAGTACGTTCGCAAGAATGAAACTCAA
AGGAATTGACGGGGACCCGCACAAGCGGTGGAGCATGTGGTTTAATTCGAAGCAACGCGAAGAACCTTACCAAGT
CTTGACATCCCTCTGACCGGCCCGTAACGGGGCCTTCCCTTCGGGGCAGAGGAGACAGGTGGTGCATGGTTGTCG
TCAGCTCGTGTCGTGAGATGTTGGGTTAAGTCCCGCAACGAGCGCAACCCCTATCCTTAGTAGCCAGCAGGTGAA
GCTGGGCACTCTAGGGAGACTGCCGGGGATAACCCGGAGGAAGGCGGGGACGACGTCAAATCATCATGCCCCTTA
TGATTTGGGCTACACACGTGCTACAATGGCGTAAACAAAGGGAAGCGAGACAGCGATGTTGAGCAAATCCCAAAA
ATAACGTCCTAGTTCGGACTGCAGTCTGCAACTCGACTGCACGAAGCTGGAATCGCTAGTAATCGCGAATCAGAA
TGTCGCGGTGAATACGTTCCCGGGTCTTGTACACACCGCCCGTCACACCATGGGAGTCAGTAACGCCCGAAGTCA
GTGACCCAACCTTACAGGAGGGAGCTGCCGAAGGCGGGACCGATAACTGGGGTGAAGTCGTAACAAGGTAGCCGT
ATCGGAAGGTGCGGCTGGATCACCTCCTTT
Strain 6
Dorea longicatena 16S ribosomal RNA coding sequence (16S rDNA)
(SEQ ID NO: 6)
AACGAGAGTTTGATCCTGGCTCAGGATGAACGCTGGCGGCGTGCTTAACACATGCAAGTCGAGCGAAGCACTTTG
GAAAGATTCTTCGGATGATTTCCTTTGTGACTGAGCGGCGGACGGGTGAGTAACGCGTGGGTAACCTGCCTCATA
CAGGGGGATAACAGTTAGAAATGACTGCTAATACCGCATAAGACCACGGTACCGCATGGTACAGTGGTAAAAACT
CCGGTGGTATGAGATGGACCCGCGTCTGATTAGGTAGTTGGTGGGGTAACGGCCTACCAAGCCGACGATCAGTAG
CCGACCTGAGAGGGTGACCGGCCACATTGGGACTGAGACACGGCCCAGACTCCTACGGGAGGCAGCAGTGGGGAA
TATTGCACAATGGAGGAAACTCTGATGCAGCGACGCCGCGTGAAGGATGAAGTATTTCGGTATGTAAACTTCTAT
CAGCAGGGAAGAAAATGACGGTACCTGACTAAGAAGCCCCGGCTAACTACGTGCCAGCAGCCGCGGTAATACGTA
GGGGGCAAGCGTTATCCGGATTTACTGGGTGTAAAGGGAGCGTAGACGGCACGGCAAGCCAGATGTGAAAGCCCG
GGGCTCAACCCCGGGACTGCATTTGGAACTGCTGAGCTAGAGTGTCGGAGAGGCAAGTGGAATTCCTAGTGTAGC
GGTGAAATGCGTAGATATTAGGAGGAACACCAGTGGCGAAGGCGGCTTGCTGGACGATGACTGACGTTGAGGCTC
GAAAGCGTGGGGAGCAAACAGGATTAGATACCCTGGTAGTCCACGCCGTAAACGATGACTGCTAGGTGTCGGGTG
GCAAAGCCATTCGGTGCCGCAGCTAACGCAATAAGCAGTCCACCTGGGGAGTACGTTCGCAAGAATGAAACTCAA
AGGAATTGACGGGGACCCGCACAAGCGGTGGAGCATGTGGTTTAATTCGAAGCAACGCGAAGAACCTTACCTGAT
CTTGACATCCCGATGACCGCTTCGTAATGGAAGCTTTTCTTCGGAACATCGGTGACAGGTGGTGCATGGTTGTCG
TCAGCTCGTGTCGTGAGATGTTGGGTTAAGTCCCGCAACGAGCGCAACCCCTATCTTCAGTAGCCAGCAGGTTAA
GCTGGGCACTCTGGAGAGACTGCCAGGGATAACCTGGAGGAAGGTGGGGATGACGTCAAATCATCATGCCCCTTA
TGACCAGGGCTACACACGTGCTACAATGGCGTAAACAAAGAGAAGCGAACTCGCGAGGGTAAGCAAATCTCAAAA
ATAACGTCTCAGTTCGGATTGTAGTCTGCAACTCGACTACATGAAGCTGGAATCGCTAGTAATCGCAGATCAGAA
TGCTGCGGTGAATACGTTCCCGGGTCTTGTACACACCGCCCGTCACACCATGGGAGTCAGTAACGCCCGAAGTCA
GTGACCCAACCGTAAGGAGGGAGCTGCCGAAGGTGGGACCGATAACTGGGGTGAAGTCGTAACAAGGTAGCCGTA
TCGGAAGGTGCGGCTGGATCACCTCCTTT
Strain 7
Clostridium innocuum 16S ribosomal RNA coding sequence (16S rDNA)
(SEQ ID NO: 7)
ATGGAGAGTTTGATCCTGGCTCAGGATGAACGCTGGCGGCATGCCTAATACATGCAAGTCGAACGAAGTTTCGAG
GAAGCTTGCTTCCAAAGAGACTTAGTGGCGAACGGGTGAGTAACACGTAGGTAACCTGCCCATGTGTCCGGGATA
ACTGCTGGAAACGGTAGCTAAAACCGGATAGGTATACAGAGCGCATGCTCAGTATATTAAAGCGCCCATCAAGGC
GTGAACATGGATGGACCTGCGGCGCATTAGCTAGTTGGTGAGGTAACGGCCCACCAAGGCGATGATGCGTAGCCG
GCCTGAGAGGGTAAACGGCCACATTGGGACTGAGACACGGCCCAAACTCCTACGGGAGGCAGCAGTAGGGAATTT
TCGTCAATGGGGGAAACCCTGAACGAGCAATGCCGCGTGAGTGAAGAAGGTCTTCGGATCGTAAAGCTCTGTTGT
AAGTGAAGAACGGCTCATAGAGGAAATGCTATGGGAGTGACGGTAGCTTACCAGAAAGCCACGGCTAACTACGTG
CCAGCAGCCGCGGTAATACGTAGGTGGCAAGCGTTATCCGGAATCATTGGGCGTAAAGGGTGCGTAGGTGGCGTA
CTAAGTCTGTAGTAAAAGGCAATGGCTCAACCATTGTAAGCTATGGAAACTGGTATGCTGGAGTGCAGAAGAGGG
CGATGGAATTCCATGTGTAGCGGTAAAATGCGTAGATATATGGAGGAACACCAGTGGCGAAGGCGGTCGCCTGGT
CTGTAACTGACACTGAGGCACGAAAGCGTGGGGAGCAAATAGGATTAGATACCCTAGTAGTCCACGCCGTAAACG
ATGAGAACTAAGTGTTGGAGGAATTCAGTGCTGCAGTTAACGCAATAAGTTCTCCGCCTGGGGAGTATGCACGCA
AGTGTGAAACTCAAAGGAATTGACGGGGGCCCGCACAAGCGGTGGAGTATGTGGTTTAATTCGAAGCAACGCGAA
GAACCTTACCAGGCCTTGACATGGAAACAAATACCCTAGAGATAGGGGGATAATTATGGATCACACAGGTGGTGC
ATGGTTGTCGTCAGCTCGTGTCGTGAGATGTTGGGTTAAGTCCCGCAACGAGCGCAACCCTTGTCGCATGTTACC
AGCATCAAGTTGGGGACTCATGCGAGACTGCCGGTGACAAACCGGAGGAAGGTGGGGATGACGTCAAATCATCAT
GCCCCTTATGGCCTGGGCTACACACGTACTACAATGGCGACCACAAAGAGCAGCGACACAGTGATGTGAAGCGAA
TCTCATAAAGGTCGTCTCAGTTCGGATTGAAGTCTGCAACTCGACTTCATGAAGTCGGAATCGCTAGTAATCGCA
GATCAGCATGCTGCGGTGAATACGTTCTCGGGCCTTGTACACACCGCCCGTCAAACCATGGGAGTCAGTAATACC
CGAAGCCGGTGGCATAACCGCAAGGAGTGAGCCGTCGAAGGTAGGACCGATGACTGGGGTTAAGTCGTAACAAGG
TATCCCTACGGGAACGTGGGGATGGATCACCTCCTTT
Strain 8
Flavonifractor plautii 16S ribosomal RNA coding sequence (16S rDNA)
(SEQ ID NO: 8)
TATTGAGAGTTTGATCCTGGCTCAGGATGAACGCTGGCGGCGTGCTTAACACATGCAAGTCGAACGGGGTGCTCA
TGACGGAGGATTCGTCCAACGGATTGAGTTACCTAGTGGCGGACGGGTGAGTAACGCGTGAGGAACCTGCCTTGG
AGAGGGGAATAACACTCCGAAAGGAGTGCTAATACCGCATGATGCAGTTGGGTCGCATGGCTCTGACTGCCAAAG
ATTTATCGCTCTGAGATGGCCTCGCGTCTGATTAGCTAGTAGGCGGGGTAACGGCCCACCTAGGCGACGATCAGT
AGCCGGACTGAGAGGTTGACCGGCCACATTGGGACTGAGACACGGCCCAGACTCCTACGGGAGGCAGCAGTGGGG
AATATTGGGCAATGGGCGCAAGCCTGACCCAGCAACGCCGCGTGAAGGAAGAAGGCTTTCGGGTTGTAAACTTCT
TTTGTCGGGGACGAAACAAATGACGGTACCCGACGAATAAGCCACGGCTAACTACGTGCCAGCAGCCGCGGTAAT
ACGTAGGTGGCAAGCGTTATCCGGATTTACTGGGTGTAAAGGGCGTGTAGGCGGGATTGCAAGTCAGATGTGAAA
ACTGGGGGCTCAACCTCCAGCCTGCATTTGAAACTGTAGTTCTTGAGTGCTGGAGAGGCAATCGGAATTCCGTGT
GTAGCGGTGAAATGCGTAGATATACGGAGGAACACCAGTGGCGAAGGCGGATTGCTGGACAGTAACTGACGCTGA
GGCGCGAAAGCGTGGGGAGCAAACAGGATTAGATACCCTGGTAGTCCACGCCGTAAACGATGGATACTAGGTGTG
GGGGGTCTGACCCCCTCCGTGCCGCAGTTAACACAATAAGTATCCCACCTGGGGAGTACGATCGCAAGGTTGAAA
CTCAAAGGAATTGACGGGGGCCCGCACAAGCGGTGGAGTATGTGGTTTAATTCGAAGCAACGCGAAGAACCTTAC
CAGGGCTTGACATCCCACTAACGAAGCAGAGATGCATTAGGTGCCCTTCGGGGAAAGTGGAGACAGGTGGTGCAT
GGTTGTCGTCAGCTCGTGTCGTGAGATGTTGGGTTAAGTCCCGCAACGAGCGCAACCCTTATTGTTAGTTGCTAC
GCAAGAGCACTCTAGCGAGACTGCCGTTGACAAAACGGAGGAAGGTGGGGACGACGTCAAATCATCATGCCCCTT
ATGTCCTGGGCCACACACGTACTACAATGGTGGTTAACAGAGGGAGGCAATACCGCGAGGTGGAGCAAATCCCTA
AAAGCCATCCCAGTTCGGATTGCAGGCTGAAACCCGCCTGTATGAAGTTGGAATCGCTAGTAATCGCGGATCAGC
ATGCCGCGGTGAATACGTTCCCGGGCCTTGTACACACCGCCCGTCACACCATGAGAGTCGGGAACACCCGAAGTC
CGTAGCCTAACCGCAAGGAGGGCGCGGCCGAAGGTGGGTTCGATAATTGGGGTGAAGTCGTAACAAGGTAGCCGT
ATCGGAAGGTGCGGCTGGATCACCTCCTTT

### ENUMERATED EMBODIMENTS

A1. A method of treating or preventing neutropenia in a subject, the method comprising administering to the subject a pharmaceutical composition comprising a purified bacterial mixture, wherein the purified bacterial mixture comprises bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A2. The method of Embodiment A1, wherein the purified bacterial mixture consists of bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A3. A method of treating or preventing neutropenia in a subject, the method comprising administering to the subject a pharmaceutical composition comprising a purified bacterial mixture, wherein the purified bacterial mixture comprises:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A4. The method of Embodiment A3, wherein the purified bacterial mixture consists of:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A5. The method of any one of Embodiments A1-A4, wherein the subject has neutropenia.
A5.1 The method of any one of Embodiments A1-A5, wherein the neutropenia is associated with cancer treatment.
A6. A method of treating or preventing a cardiovascular disease in a subject, the method comprising administering to the subject a pharmaceutical composition comprising a purified bacterial mixture, wherein the purified bacterial mixture comprises bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A7. The method of Embodiment A6, wherein the purified bacterial mixture consists of bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A8. A method of treating or preventing a cardiovascular disease in a subject, the method comprising administering to the subject a pharmaceutical composition comprising a purified bacterial mixture, wherein the purified bacterial mixture comprises:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A9. The method of Embodiment A8, wherein the purified bacterial mixture consists of:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A10. The method of any one of Embodiments A6-A9, wherein the subject has atherosclerosis.
A10.1 The method of any one of Embodiments A6-A9, wherein the subject has had a stroke.
A11. A method of treating or preventing asthma in a subject, the method comprising administering to the subject a pharmaceutical composition comprising a purified bacterial mixture, wherein the purified bacterial mixture comprises bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A12. The method of Embodiment A11, wherein the purified bacterial mixture consists of bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A13. A method of treating or preventing asthma in a subject, the method comprising administering to the subject a pharmaceutical composition comprising a purified bacterial mixture, wherein the purified bacterial mixture comprises:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A14. The method of Embodiment A13, wherein the purified bacterial mixture consists of:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A15. The method of any one of Embodiments A11-A14, wherein the subject has asthma.
A16. A method of treating or preventing cancer in a subject, the method comprising administering to the subject a pharmaceutical composition comprising a purified bacterial mixture, wherein the purified bacterial mixture comprises bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A17. The method of Embodiment A16, wherein the purified bacterial mixture consists of bacterial strains of species Clostridium bolteae, Anaerotruncus colihominis, Drancourtella massiliensis, Clostridium symbiosum, Blautia producta, Dorea longicatena, Clostridium innocuum, and Flavonifractor plautii.
A18. A method of treating or preventing cancer in a subject, the method comprising administering to the subject a pharmaceutical composition comprising a purified bacterial mixture, wherein the purified bacterial mixture comprises:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A19. The method of Embodiment A18, wherein the purified bacterial mixture consists of:
   (i) a bacterial strain comprising a 16S rDNA sequence with at least 97% sequence identity to the nucleic acid sequence of SEQ ID NO: 1;
   (ii) a bacterial strain comprising a 16S rDNA sequence with at least 97% sequence identity to the nucleic acid sequence of SEQ ID NO: 2;
   (iii) a bacterial strain comprising a 16S rDNA sequence with at least 97% sequence identity to the nucleic acid sequence of SEQ ID NO: 3;
   (iv) a bacterial strain comprising a 16S rDNA sequence with at least 97% sequence identity to the nucleic acid sequence of SEQ ID NO: 4;
   (v) a bacterial strain comprising a 16S rDNA sequence with at least 97% sequence identity to the nucleic acid sequence of SEQ ID NO: 5;
   (vi) a bacterial strain comprising a 16S rDNA sequence with at least 97% sequence identity to the nucleic acid sequence of SEQ ID NO: 6;
   (vii) a bacterial strain comprising a 16S rDNA sequence with at least 97% sequence identity to the nucleic acid sequence of SEQ ID NO: 7; and
   (viii) a bacterial strain comprising a 16S rDNA sequence with at least 97% sequence identity to the nucleic acid sequence of SEQ ID NO: 8.
A20. The method of any one of Embodiments A16-A19, wherein the subject has cancer.
A21. The method of any one of Embodiments A16-A20, wherein the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.
A22. The method of any one of the preceding Embodiments, wherein the purified bacterial mixture comprises at least 9, at least 10, at least 11, at least 12, at least 13, at least 14, at least 15, at least 16, at least 17, at least 18, at least 19, or at least 20 bacterial strains.
A23. The method of any one of the preceding Embodiments, wherein the purified bacterial mixture comprises 8-20, 8-15, or 8-10 bacterial strains.
C1. The method of any one of the preceding Embodiments, wherein the pharmaceutical composition induces neutrophil generation in the subject.
C2. The method of any one of the preceding Embodiments, wherein the pharmaceutical composition induces production of a protein selected from the group consisting of IL-5, IL-9, IL-10, IL-12(p70), IL20, IL-23, IL-28A, IL-33, thymic stromal lymphopoietin (TSLP), thrombopoietin (TPO), macrophage inflammatory protein-1 alpha (MIP-1a), monocyte chemotactic protein 3 (MCP3), leukemia inhibitory factor (LIF), interferon-gamma (IFN-γ), fractalkine, and eotaxin-3, in the subject.
C2.1 The method of any one of the preceding Embodiments, wherein the pharmaceutical composition induces production of IL-5 in the subject.
C2.2 The method of any one of the preceding Embodiments, wherein the pharmaceutical composition induces production of IL-9 in the subject.
C2.3 The method of any one of the preceding Embodiments, wherein the pharmaceutical composition induces production of IL-33 in the subject.
C2.4 The method of any one of the preceding Embodiments, wherein the pharmaceutical composition induces production of G-CSF in the subject.
C3. The method of any one of the preceding Embodiments, wherein the pharmaceutical composition induces proliferation and/or accumulation of T regulatory cells (Tregs) in the subject.
C3.1 The method of Embodiment C3, wherein the Tregs are ST2+ Tregs.
C3.2 The method of any one of the preceding Embodiments, wherein the pharmaceutical composition promotes tissue repair.
C4. The method of any one of the preceding Embodiments, wherein the bacterial strains originate from more than one human donor.
C5. The method of any one of Embodiments A1-C4, wherein the bacterial strains are lyophilized.
C6. The method of any one of Embodiments A1-C4, wherein the bacterial strains are spray-dried.
C7. The method of any one of Embodiments A1-C6, wherein one or more of the bacterial strains are in spore form.
C8. The method of any one of Embodiments A1-C7, wherein each of the bacterial strains is in spore form.
C9. The method of any one of Embodiments A1-C6, wherein one or more of the bacterial strains are in vegetative form.
C10. The method of any one of Embodiments A1-C6 or C9, wherein each of the bacterial strains is in vegetative form.
D1. The method of any one of the preceding Embodiments, wherein the pharmaceutical composition further comprises a pharmaceutically acceptable excipient.
D2. The method of Embodiment D1, wherein the pharmaceutical composition is formulated for oral delivery.
D3. The method of Embodiment D1, wherein the pharmaceutical composition is formulated for rectal delivery.
D4. The method of any one of Embodiments D1-D3, wherein the pharmaceutical composition is formulated for delivery to the intestine.
D5. The method of any one of Embodiments D1-D4, wherein the pharmaceutical composition is formulated for delivery to the colon.
D6. The method of any one of Embodiments D1-D5, wherein the pharmaceutical composition comprises one or more enteric polymers.
D7. The method of any one of Embodiments D1-D6, wherein the pharmaceutical composition is in a capsule.
D8. The method of Embodiment D7, wherein the capsule comprises 1×10⁷ to 1×10¹⁰ colony-forming units (CFUs) of each of the bacterial strains.
E1. The method of any one of the preceding Embodiments, wherein the pharmaceutical composition is administered as one dose.
E2. The method of any one of the preceding Embodiments, wherein the pharmaceutical composition is administered as multiple doses.
E3. The method of any one of the preceding Embodiments, wherein each dose of the pharmaceutical composition comprises administration of multiple capsules.
E4. The method of any one of the preceding Embodiments, wherein each dose of the pharmaceutical composition comprises about 1.1×10¹¹ colony-forming units (CFUs) of the bacterial strains.
E5. The method of any one of the preceding Embodiments, wherein each dose of the pharmaceutical composition comprises about 2.2×10¹⁰ colony-forming units (CFUs) of the bacterial strains.
E6. The method of any one of the preceding Embodiments, wherein the subject is administered an antibiotic prior to administration of the composition.
E7. The method of Embodiment E6, wherein the antibiotic is selected from the group consisting of vancomycin kanamycin, gentamicin, colistin, metronidazole, clindamycin, fidaxomicin, penicillin, streptomycin, and cefoperazone.
E8. The method of Embodiment E6 or E7, wherein the antibiotic is vancomycin.
E9. The method of any one of the preceding Embodiments, wherein the subject is a human.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

### Example 1: Evaluation of an 8-Strain Defined Bacterial Mixture in a Double-Blind Placebo-Controlled Phase 2 Study for Prevention of Recurrent C. difficile Infection

### 1. Summary

Fecal microbiota transplantation (FMT) shows promising clinical success in treating patients with recurrent C. difficile infection (rCDI); however, the composition and quality attributes of donor-derived procedures are inherently variable. VE303 is a first-in-class, rationally defined consortium consisting of 8 purified, clonal strains belonging to Clostridium clusters IV, XIVa, and XVII. In the Phase 2 CONSORTIUM Study, VE303 had an acceptable safety profile, with a lower CDI recurrence rate in the VE303 high-dose group (13.8%) than in the placebo group (45.5%). Higher VE303 strain colonization was associated with clinical efficacy and VE303 strains promoted early restoration of the microbiota. Here, we examined if VE303 was associated with reduced inflammation in patients at high risk of CDI.

### 2. Methods

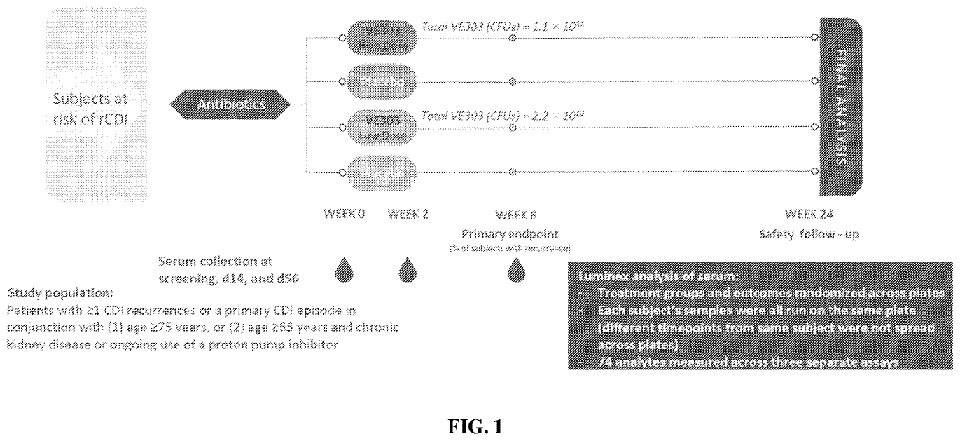

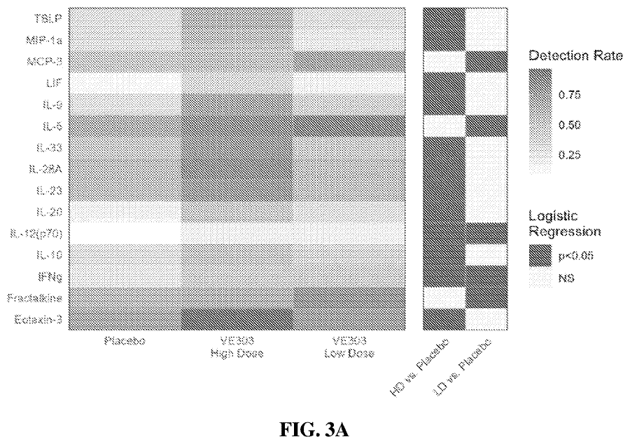

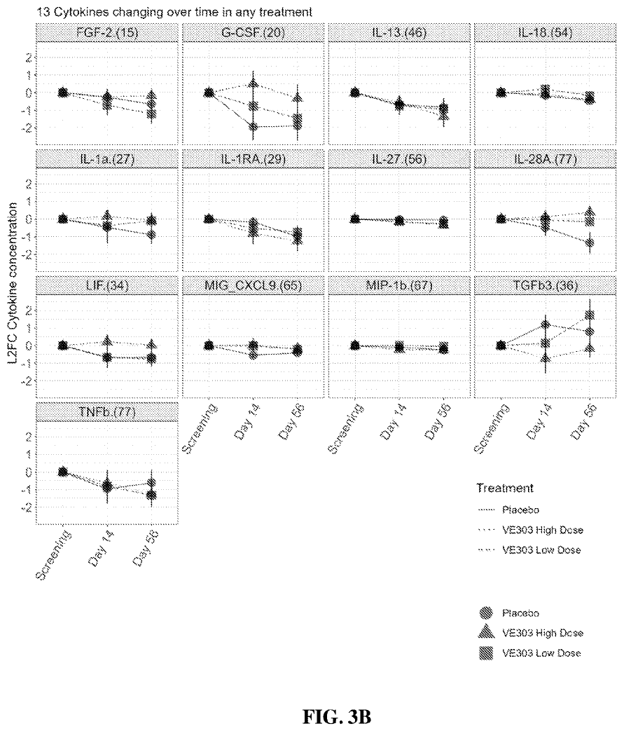

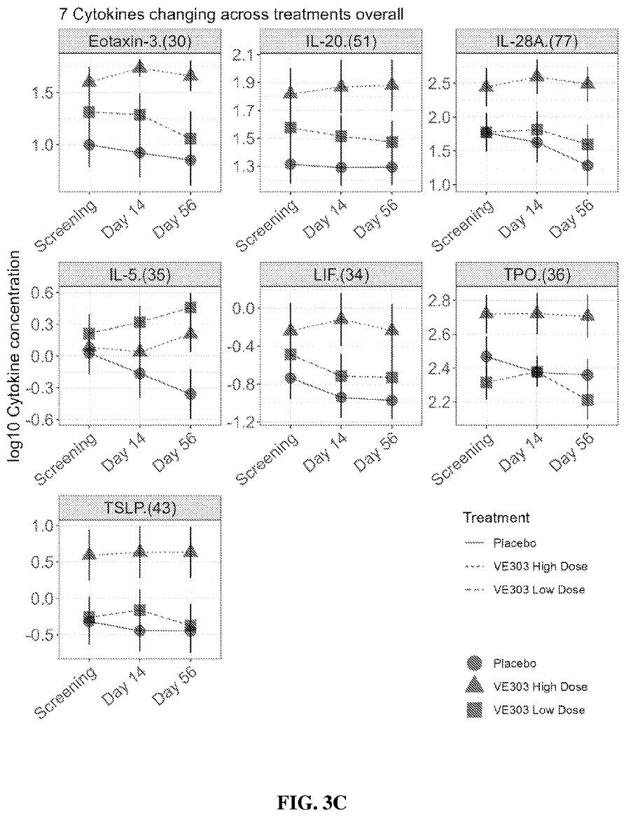

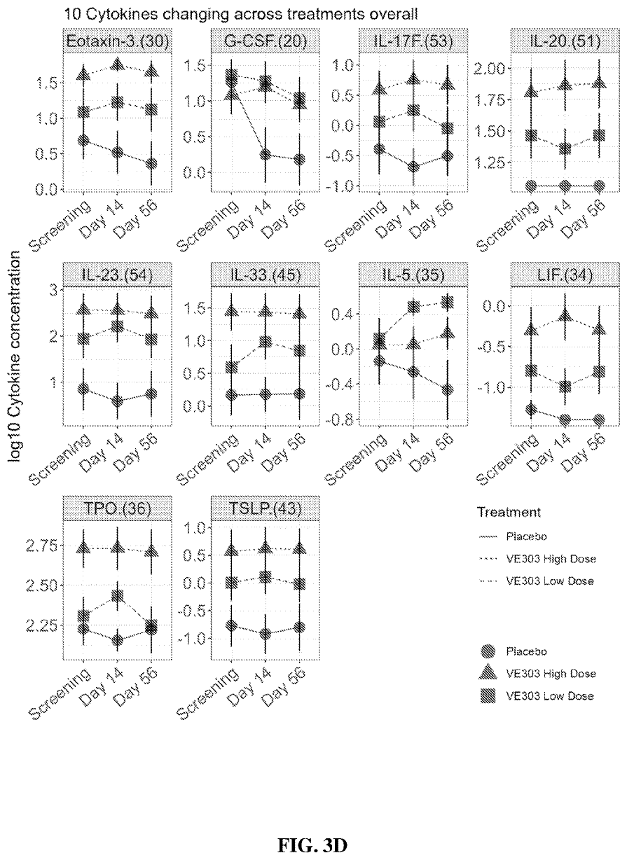

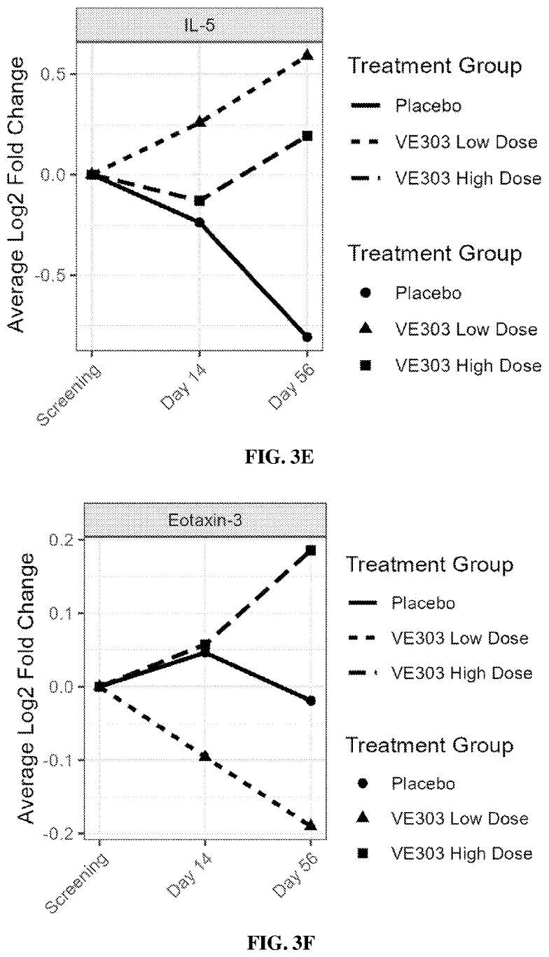

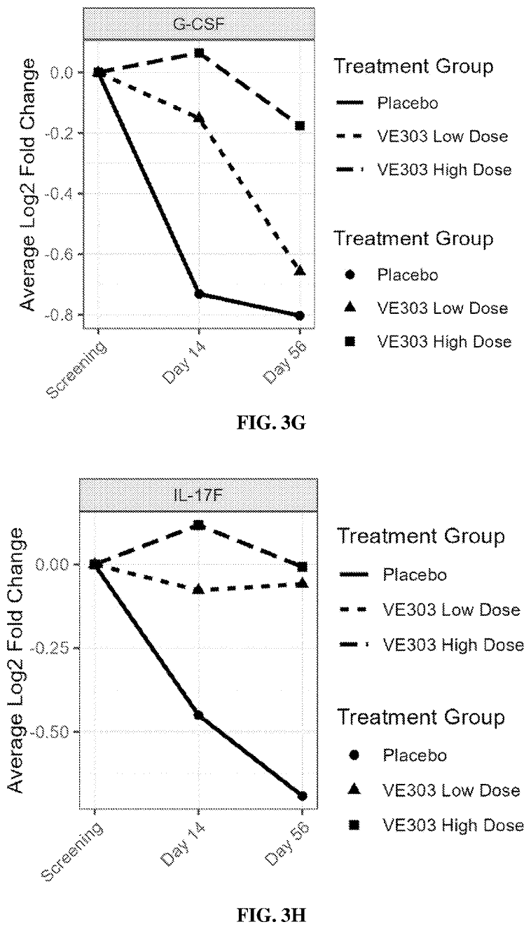

The CONSORTIUM Study was a double-blind, placebo-controlled phase 2 trial. After completing standard-of-care antibiotic treatment for a laboratory-confirmed CDI episode, 80 subjects were randomized to 1 of 3 groups and received low-dose VE303 (2.2×10¹⁰ total CFU), high-dose VE303 (1.1×10¹¹ total CFU), or placebo orally once daily for 14 days (FIG. 1). Subjects were followed for 24 weeks to monitor safety, rCDI episodes, and immune biomarkers. Fecal calprotectin was measured on days 1, 14, 28, and 56 following the start of treatment (FIGS. 2A-2C). Serum cytokines were measured at screening, day 14, and day 56 (FIGS. 3A-3H).

### 3. Results

Increased fecal calprotectin indicated higher inflammation in subjects with an on-study recurrence compared to non-recurrent subjects across treatment groups (FIGS. 2A-2D, p<0.05, Linear mixed effects [LME]). Additionally, fecal calprotectin increased over time from Day 1 in nonrecurrent placebo recipients (p<0.05, LME) but not in nonrecurrent subjects in either VE303 group (p>0.2, LME) (FIGS. 2A-2D). Fecal calprotectin was significantly correlated with multiple cytokines and chemokines, including eotaxin, G-CSF, CXCL1, IL-1RA, IL-4, IL-5, IL-6, IL-8, CXCL9, and TNF-α (FIG. 2E).

Cytokines involved in tissue repair, including IL-33, IL-9, and IL-28A, were detected more frequently in VE303 high-dose compared with placebo subjects (FIG. 3A, Logistic Regression, p<0.05). Finally, treatment-specific changes were observed for a subset of cytokines (FIGS. 3A-3D). Over the course of the study, subjects administered VE303 exhibited significantly higher levels of TSLP, MIP-1α, MCP-3, LIF, IL-9, IL-33, IL-28A, IL-23, IL-20, IL-12(p70), IL-10, IFN-γ, fractalkine, and eotaxin-3, compared to subjects administered placebo (FIG. 3A). Peripheral concentrations of multiple cytokines changed over time between treatment groups, including LIF, IL-1α, IL-27, TGF-03, IL-1RA, TNF-0, MIP-10, CXCL9, IL-13, LIF, G-CSF, IL-18, and FGF-2 (FIG. 3B). At day 14 post-administration, subjects administered high doses of VE303 exhibited elevated levels of eotaxin-3 (compared to placebo), and elevated levels of eotaxin-3, IL-20, IL-28A, IL-5, LIF, TPO, and TSLP (FIG. 3C). Among non-recurrent subjects, at day 14 post-administration, subjects administered VE303 exhibited higher levels of eotaxin-3, G-CSF, IL-17F, IL-20, TPO, TSLP, LIF, and IL-33, compared to subjects administered placebo (FIG. 3D). At day 56 post-administration, subjects administered VE303 exhibited higher levels of eotaxin-3, IL-23, IL-20, TPO, TSLP, and IL-33, compared to subjects administered placebo (FIG. 3D). G-CSF (FIGS. 3B and 3G), IL-17F (FIG. 3H), and IL-28A (FIG. 3B) were maintained in the VE303 high-dose group but declined in the placebo group. Additionally, VE303 promoted stable or increased concentrations of cytokines associated with tissue repair, including IL-5 (FIG. 3E) and eotaxin-3 (FIG. 3F).

### 4. Conclusions

In subjects at high risk of rCDI, VE303 dosing was associated with reduced inflammation determined by lower fecal calprotectin and changes in circulating serum cytokines. VE303 may provide benefits beyond microbiome restoration in patients with CDI by limiting pathological inflammation.

What is claimed is:

1. A method of treating or preventing recurrent Clostridium difficile infection (rCDI) in a subject, the method comprising administering to the subject a composition comprising a purified bacterial mixture, the purified bacterial mixture comprising:
(i) a bacterial strain belonging to Clostridium bolteae;
(ii) a bacterial strain belonging to Anaerotruncus colihominis;
(iii) a bacterial strain belonging to Sellimonas intestinalis;
(iv) a bacterial strain belonging to Clostridium symbiosum;
(v) a bacterial strain belonging to Blautia producta;
(vi) a bacterial strain belonging to Dorea longicatena;
(vii) a bacterial strain belonging to Clostridium innocuum; and
(viii) a bacterial strain belonging to Flavonifractor plautii,
wherein a concentration of fecal calprotectin in a sample of the subject is not substantially increased for at least 1 week after administration of the composition, relative to a baseline concentration of fecal calprotectin prior to administration of the composition.

2. A method of treating or preventing recurrent Clostridium difficile infection (rCDI) in a subject, the method comprising administering to the subject a composition comprising a purified bacterial mixture, the purified bacterial mixture comprising:
(i) a bacterial strain comprising a 16S rDNA sequence with at least 97% sequence identity to the nucleotide sequence of SEQ ID NO: 1;
(ii) a bacterial strain comprising a 16S rDNA sequence with at least 97% sequence identity to the nucleotide sequence of SEQ ID NO: 2;
(iii) a bacterial strain comprising a 16S rDNA sequence with at least 97% sequence identity to the nucleotide sequence of SEQ ID NO: 3;
(iv) a bacterial strain comprising a 16S rDNA sequence with at least 97% sequence identity to the nucleotide sequence of SEQ ID NO: 4;
(v) a bacterial strain comprising a 16S rDNA sequence with at least 97% sequence identity to the nucleotide sequence of SEQ ID NO: 5;
(vi) a bacterial strain comprising a 16S rDNA sequence with at least 97% sequence identity to the nucleotide sequence of SEQ ID NO: 6;
(vii) a bacterial strain comprising a 16S rDNA sequence with at least 97% sequence identity to the nucleotide sequence of SEQ ID NO: 7; and
(viii) a bacterial strain comprising a 16S rDNA sequence with at least 97% sequence identity to the nucleotide sequence of SEQ ID NO: 8,
wherein a concentration of fecal calprotectin in a sample of the subject is not substantially increased for at least 1 week after administration of the composition, relative to a baseline concentration of fecal calprotectin prior to administration of the composition.

3. The method of any one of the preceding claims, wherein the concentration of fecal calprotectin in the sample of the subject does not exceed 140%, 130%, 120%, or 110% of the baseline concentration of fecal calprotectin in the subject.

4. The method of any one of the preceding claims, wherein the concentration of fecal calprotectin is not substantially increased over a period of at least 2, 3, 4, 5, 6, 7, or 8 weeks following administration of the composition.

5. The method of any one of the preceding claims, wherein the baseline concentration of fecal calprotectin is a fecal calprotectin concentration in a baseline sample of the subject obtained 48 hours or fewer prior to administration of the composition.

6. The method of any one of the preceding claims, wherein the method further comprises measuring the concentration of fecal calprotectin in one or more samples of the subject after administration of the composition.

7. The method of any one of the preceding claims, wherein the method further comprises identifying the subject as having an elevated concentration of fecal calprotectin, if the level of fecal calprotectin is increased relative to the baseline concentration, and administering one or more additional doses of the composition to the subject.

8. The method of claim 7, wherein the elevated concentration of fecal calprotectin is at least 150%, at least 175%, at least 200%, at least 250%, or at least 300% relative to the baseline concentration of fecal calprotectin in the subject.

9. A method of treating and/or preventing a condition in a subject, the method comprising administering to the subject a composition comprising a purified bacterial mixture, the purified bacterial mixture comprising:
(i) a bacterial strain belonging to Clostridium bolteae;
(ii) a bacterial strain belonging to Anaerotruncus colihominis;
(iii) a bacterial strain belonging to Sellimonas intestinalis;
(iv) a bacterial strain belonging to Clostridium symbiosum;
(v) a bacterial strain belonging to Blautia producta;
(vi) a bacterial strain belonging to Dorea longicatena;
(vii) a bacterial strain belonging to Clostridium innocuum; and
(viii) a bacterial strain belonging to Flavonifractor plautii,
wherein the condition is selected from the group consisting of neutropenia, cardiovascular disease, asthma, and cancer.

10. A method of treating and/or preventing a condition in a subject, the method comprising administering to the subject a composition comprising a purified bacterial mixture, the purified bacterial mixture comprising:
(i) a bacterial strain comprising a 16S rDNA sequence with at least 97% sequence identity to the nucleotide sequence of SEQ ID NO: 1;
(ii) a bacterial strain comprising a 16S rDNA sequence with at least 97% sequence identity to the nucleotide sequence of SEQ ID NO: 2;
(iii) a bacterial strain comprising a 16S rDNA sequence with at least 97% sequence identity to the nucleotide sequence of SEQ ID NO: 3;
(iv) a bacterial strain comprising a 16S rDNA sequence with at least 97% sequence identity to the nucleotide sequence of SEQ ID NO: 4;
(v) a bacterial strain comprising a 16S rDNA sequence with at least 97% sequence identity to the nucleotide sequence of SEQ ID NO: 5;
(vi) a bacterial strain comprising a 16S rDNA sequence with at least 97% sequence identity to the nucleotide sequence of SEQ ID NO: 6;
(vii) a bacterial strain comprising a 16S rDNA sequence with at least 97% sequence identity to the nucleotide sequence of SEQ ID NO: 7; and
(viii) a bacterial strain comprising a 16S rDNA sequence with at least 97% sequence identity to the nucleotide sequence of SEQ ID NO: 8,
wherein the condition is selected from the group consisting of neutropenia, cardiovascular disease, asthma, and cancer.

11. The method of claim 9 or 10, wherein the subject has neutropenia.

12. The method of claim 11, wherein the neutropenia results from cancer treatment.

13. The method of any one of claims 9-12, wherein the subject has a cardiovascular disease.

14. The method of any one of claims 9-13, wherein the subject has atherosclerosis.

15. The method of any one of claims 9-14, wherein the subject has had a stroke.

16. The method of any one of claims 9-15, wherein the subject has asthma.

17. The method of any one of claims 9-16, wherein the subject has cancer.

18. The method of claim 17, wherein the cancer is selected from the group consisting of melanoma, squamous cell cancer, small-cell lung cancer, non-small cell lung cancer, lung cell adenocarcinoma, squamous lung cell carcinoma, peritoneal cancer, hepatocellular cancer, gastrointestinal cancer, esophageal cancer, stomach cancer, pancreatic cancer, glioblastoma, cervical cancer, ovarian cancer, liver cancer, bladder cancer, hepatoma, breast cancer, colon cancer, colorectal cancer, endometrial carcinoma, uterine carcinoma, salivary gland carcinoma, kidney cancer, prostate cancer, vulval cancer, thyroid cancer, hepatic carcinoma, gastric cancer, head-and-neck cancer, leukemia, and lymphoma.

19. The method of any one of the preceding claims, wherein the composition induces neutrophil differentiation in the subject.

20. The method of any one of the preceding claims, wherein the composition increases circulating neutrophil abundance in the subject.

21. The method of any one of the preceding claims, wherein the composition inhibits infiltration of neutrophils into an intestinal lumen of the subject.

22. The method of any one of the preceding claims, wherein the composition induces proliferation and/or accumulation of regulatory T cells (Tregs) in the subject.

23. The method of claim 22, wherein the Tregs are ST2+ Tregs.

24. The method of any one of the preceding claims, wherein the composition promotes tissue repair in the subject.

25. The method of any one of the preceding claims, wherein the composition induces production of a protein selected from the group consisting of calprotectin, IL-5, IL-9, IL-10, IL-12(p70), IL-20, IL-23, IL-28A, IL-33, thymic stromal lymphopoietin (TSLP), thrombopoietin (TPO), macrophage inflammatory protein-1 alpha (MIP-1α), monocyte chemotactic protein 3 (MCP3), leukemia inhibitory factor (LIF), interferon-gamma (IFN-γ), fractalkine, and eotaxin-3, in the subject.

26. The method of any one of the preceding claims, wherein the composition induces production of calprotectin in the subject.

27. The method of any one of the preceding claims, wherein the purified bacterial mixture consists of:
(i) a bacterial strain comprising a 16S rDNA sequence with at least 99% sequence identity to the nucleotide sequence of SEQ ID NO: 1;
(ii) a bacterial strain comprising a 16S rDNA sequence with at least 99% sequence identity to the nucleotide sequence of SEQ ID NO: 2;
(iii) a bacterial strain comprising a 16S rDNA sequence with at least 99% sequence identity to the nucleotide sequence of SEQ ID NO: 3;
(iv) a bacterial strain comprising a 16S rDNA sequence with at least 99% sequence identity to the nucleotide sequence of SEQ ID NO: 4;
(v) a bacterial strain comprising a 16S rDNA sequence with at least 99% sequence identity to the nucleotide sequence of SEQ ID NO: 5;
(vi) a bacterial strain comprising a 16S rDNA sequence with at least 99% sequence identity to the nucleotide sequence of SEQ ID NO: 6;
(vii) a bacterial strain comprising a 16S rDNA sequence with at least 99% sequence identity to the nucleotide sequence of SEQ ID NO: 7; and
(viii) a bacterial strain comprising a 16S rDNA sequence with at least 99% sequence identity to the nucleotide sequence of SEQ ID NO: 8.

28. The method of any one of the preceding claims, wherein the bacterial strains are lyophilized or spray-dried.

29. The method of any one of the preceding claims, wherein one or more of the bacterial strains are in spore form.

30. The method of any one of the preceding claims, wherein the composition is a pharmaceutical composition comprising the purified bacterial mixture and a pharmaceutically acceptable excipient.

31. The method of claim 30, wherein the pharmaceutical composition is administered orally.

32. The method of claim 30, wherein the pharmaceutical composition is administered rectally.

33. The method of any one of claims 30-32, wherein the pharmaceutical composition is formulated for delivery to the intestine.

34. The method of any one of claims 30-33, wherein the pharmaceutical composition is formulated for delivery to the colon.

35. The method of any one of claims 30-34, wherein the pharmaceutical composition comprises one or more enteric polymers.

36. The method of any one of claims 30-35, wherein the pharmaceutical composition is administered to the subject as multiple doses over multiple days.

37. The method of claim 36, wherein the subject is administered about 1.6×10⁹ colony-forming units (CFUs) of the bacterial strains per day.

38. The method of any one of claims 30-37, wherein the subject is administered a total of about 2.2×10¹⁰ colony-forming units (CFUs) of the bacterial strains.

39. The method of claim 36, wherein the subject is administered about 8.0×10⁹ colony-forming units (CFUs) of the bacterial strains per day.

40. The method of any one of claims 30-37, or 39, wherein the subject is administered about 1×10¹¹ colony-forming units (CFUs) of the bacterial strains.

41. The method of any one of claims 30-40, wherein the method comprises administering an antibiotic to the subject prior to administration of the pharmaceutical composition.

42. The method of claim 41, wherein the antibiotic is vancomycin.

43. The method of any one of the preceding claims, wherein the subject is a human.


SEQUENCE LISTING (8 sequences)

SEQ ID NO: 1 - DNA, 1530 bp, Clostridium bolteae
  source 1..1530: mol_type = genomic DNA
atgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaggagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 2 - DNA, 1521 bp, Anaerotruncus colihominis
  source 1..1521: mol_type = genomic DNA
caaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 3 - DNA, 1528 bp, unidentified
  source 1..1528: mol_type = genomic DNA; note = Sellimonas intestinalis
acgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctccttt

SEQ ID NO: 4 - DNA, 1527 bp, Clostridium symbiosum
  source 1..1527: mol_type = genomic DNA
atgagagtttgatcctggctcaggatgaacgctggcggcgtgcctaacacatgcaagtcgaacgaagcgatttaacggaagttttcggatggaagttggattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctccttt

SEQ ID NO: 5 - DNA, 1530 bp, Blautia producta
  source 1..1530: mol_type = genomic DNA
tcagagagtttgatcctggctcaggatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacatcagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcctagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggagggagctgccgaaggcgggaccgataactggggtgaagtcgtaacaaggtagccgtatcggaaggtgcggctggatcacctccttt

SEQ ID NO: 6 - DNA, 1529 bp, Dorea longicatena
  source 1..1529: mol_type = genomic DNA
aacgagagtttgatcctggctcaggatgaacgctggcggcgtgcttaacacatgcaagtcgagcgaagcactttggaaagattcttcggatgatttcctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 7 - DNA, 1537 bp, Clostridium innocuum
  source 1..1537: mol_type = genomic DNA
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgcaaggagtgagccgtcgaaggtaggaccgatgactggggttaagtcgtaacaaggtatccctacgggaacgtggggatggatcacctccttt

SEQ ID NO: 8 - DNA, 1530 bp, Flavonifractor plautii
  source 1..1530: mol_type = genomic DNA
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt